(19) United States
(12) Patent Application Publication
(10) Pub. No.: US 2024/0312578 A1
(43) Pub. Date: Sep. 19, 2024

(54) SYSTEM, SERVER DEVICE, AND ELECTRONIC DEVICE, AND RELATED METHODS FOR DISEASE MONITORING AND/OR HANDLING

(71) Applicant: Nordsjællands Hospital - Hillerød, Hillerød (DK)

(72) Inventor: Thea Kølsen FISCHER, Hillerød (DK)

(21) Appl. No.: 18/576,421

(22) PCT Filed: Jul. 5, 2022
(86) PCT No.: PCT/EP22/68532

§ 371 Date: Jan. 4, 2024

(30) Foreign Application Priority Data
Jul. 5, 2021 (EP) 21183678.8

(51) Int. Cl. G16H 10/60; G16H 10/20

(52) U.S. Cl. CPC G16H 10/60; G16H 10/20

(57) ABSTRACT

A system for disease monitoring and/or handling is disclosed. The system comprises an electronic device and a server device. The system is configured to obtain a hospitalization status of a patient. The system is configured to obtain first patient data of the patient comprising patient status data and symptom data. The patient status data is indicative of one or more of temperature, pulse, respiratory rate, oxygen saturation, blood pressure, consciousness, and blood sugar of the patient. The symptom data is indicative of one or more symptoms of the patient. The system is configured to determine a first patient metric based on the first patient data. The system is configured to determine whether the first patient metric satisfies a first criterion. The system is configured in accordance with the first patient metric satisfying the first criterion, to output a first action request.

The present disclosure relates to a system, a server device, and an electronic device for disease monitoring and/or handling and related methods. In particular, the present disclosure relates to system, server device, electronic device, and methods which facilitate tracking, monitoring, and handling of a disease and in particular virus diseases for example caused by Corona virus or Influenza virus.

### BACKGROUND

Infectious diseases, such as epidemic and pandemics, continue to pose a public health challenge and are a threat to the capacity limits of our healthcare systems, and remain a worldwide problem. It is challenging to limit the spread of a virus/disease and to monitor whether people are infected or not and in particular whether to spend resources on diagnostic tests and/or treatment. Aligned with the epidemic challenges, are the general challenges regarding the hospital capacity to handle an aging population. People generally live longer and with that also comes a constant growing prevalence of chronic diseases and need for care. The number of hospital beds on the other hand is constantly being reduced for reducing the costs in the healthcare system. Therefore new strategies are needed for monitoring and/or handling diseases and patients.

### SUMMARY

Accordingly, there is a need for a method and devices which improve the monitoring and/or handling of diseases and that provides a tool for limiting the spreading and/or handling of a disease.

A system for disease monitoring and/or handling is disclosed. The system comprises an electronic device and a server device. The system is configured to obtain a hospitalization status of a patient. The system is configured to obtain first patient data of the patient comprising patient status data and symptom data. The patient status data is indicative of one or more of temperature, pulse, respiratory rate, oxygen saturation, blood pressure, consciousness, and blood sugar of the patient. The symptom data is indicative of one or more symptoms of the patient. The system is configured to determine a first patient metric based on the first patient data. The system is configured to determine whether the first patient metric satisfies a first criterion. The system is configured in accordance with the first patient metric satisfying the first criterion, to output a first action request.

Also disclosed is a server device for disease monitoring and/or handling. The server device comprises a memory, an interface, and one or more processors. The one or more processors are configured to obtain a hospitalization status of a patient. The one or more processors are configured to obtain first patient data of the patient comprising patient status data and symptom data from an electronic device. The patient status data is indicative of one or more of temperature, pulse, respiratory rate, oxygen saturation and blood pressure of the patient. The symptom data is indicative of one or more symptoms of the patient. The one or more processors are configured to determine a first patient metric based on the first patient data. The one or more processors are configured to determine whether the first patient metric satisfies a first criterion. The one or more processors are configured in accordance with the first patient metric satisfying the first criterion, to transmit a first action request to the electronic device.

Also disclosed is an electronic device for disease monitoring and/or handling. The electronic device comprises a memory; an interface; and a processor. The processor is configured to obtain a hospitalization status of a patient from a server device. The processor is configured to obtain first patient data of the patient comprising patient status data and symptom data via the interface. The patient status data is indicative of one or more of temperature, pulse, respiratory rate, oxygen saturation, and blood pressure of the patient. The symptom data is indicative of one or more symptoms of the patient. The processor is configured to transmit the first patient data to the server device. The processor is configured to obtain a first action request from the server device. The processor is configured to output the first action request via the interface.

Also disclosed is a method for disease monitoring and/or handling. The method comprises obtaining a hospitalization status of a patient. The method comprises obtaining first patient data of the patient comprising patient status data and symptom data. The patient status data is indicative of one or more of temperature, pulse, respiratory rate, oxygen saturation, and blood pressure of the patient. The symptom data is indicative of one or more symptoms of the patient. The method comprises determining a first patient metric based on the first patient data. The method comprises determining whether the first patient metric satisfies a first criterion. The method comprises in accordance with the first patient metric satisfying the first criterion, outputting a first action request.

The present disclosure provides an improved monitoring and/or handling of diseases. The present disclosure provides a tool for avoiding the saturation of hospital capacities and resources and provides a tool for limiting the spread and/or handling of a disease. Further, the present disclosure facilitates improved use and/or utilization of hospital resources in order to allow for hospitalization of people being in a critical condition (for example needing advanced medical equipment only available in hospitals), while home hospitalizing people e.g. being in a less critical condition. This allows to focus the hospital resources on the people being at high risk e.g. the people in need of hospital treatment. This may limit the number of death caused by a disease and limit the spreading of a disease in hospitals.

It is an advantage of the present disclosure that users/patients may be able to monitor their patient status (such as user condition, e.g. health condition) and/or symptoms, locally, e.g. on an electronic device, without having to go to a hospital (such as hospitalized) or a doctor. A user may be able to be home hospitalized and still be monitored by health care professionals/personnel (such as caretakers, doctors, and nurses). Furthermore, users/patients are able to provide information about their health condition in an easy and flexible way, e.g. without having to leave their home but merely by using their electronic device. This may for example provide that hospitals and health care facilities are not overloaded in periods where a pandemic or epidemic are present.

It is also an advantage of the present disclosure that users/patients that may be in a high risk category and/or users/patients that may be infected with the disease can be guided on how to take precautionary measures. Further, the present disclosure provides an effective collection of health data, patient data, patient status data, and/or symptom data, and in turn resulting in an improved foundation for modelling and understanding a disease.

Also, the present disclosure may give guidance/advice to users/patients in relation to which actions that should be taken depending on their patient data and user condition. For example, the present disclosure may provide guidance to users/patients in relation to how to handle their user condition, e.g. when a pandemic or epidemic are present.

### BRIEF DESCRIPTION OF THE DRAWINGS

The above and other features and advantages of the present invention will become readily apparent to those skilled in the art by the following detailed description of exemplary embodiments thereof with reference to the attached drawings, in which:

FIG. 1 schematically illustrates an exemplary system for disease monitoring and/or handling according to the present disclosure,

FIGS. 2A-B is a flow diagram of an exemplary method according to the present disclosure,

FIG. 3 is a flow diagram illustrating an example on how to obtain first patient data and second data, such as second primary data and/or second secondary data, and

FIG. 4 schematically illustrates a user interface of an electronic device according to the present disclosure.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

A system for disease monitoring and/or handling is disclosed. The system comprises an electronic device and a server device. The electronic device may comprise a memory, an interface (such as one or more interfaces), and a processor (such as one or more processors). The memory circuitry may be one or more of a buffer, a flash memory, a hard drive, a removable media, a volatile memory, a non-volatile memory, a random access memory (RAM), or other suitable device. In a typical arrangement, the memory circuitry may include a non-volatile memory for long term data storage and a volatile memory that functions as system memory for the processor circuitry. The memory circuitry may exchange data with the processor circuitry over a data bus. Control lines and an address bus between the memory circuitry and the processor circuitry also may be present. The memory circuitry is considered a non-transitory computer readable medium. The memory circuitry may be configured to store sensor data, and mobility data in a part of the memory.

The interface may comprise a wired interface and/or a wireless interface. The interface may comprise one or more displays including a first display.

The electronic device may be configured to act as a user device that a user/patient may use for disease monitoring and/or handling. The electronic device may comprise a computer device (such as a mobile phone, a smartphone, a cell phone, a tablet, and/or a computer such as a laptop or a stationary computer).

The server device may comprise a memory, one or more interfaces, and one or more processors. The server device may be configured to communicate with the electronic device, e.g. via a network, such as a global network, e.g. the internet.

The disease to be handled/monitored may be an epidemic, a pandemic, or any other disease that may be transmitted and spread between human beings via a virus and/or bacteria. The disease may for example be Covid-19, Influenza, Ebola, and/or plague. In other words, a system for epidemic or pandemic monitoring and/or handling of disease and spreading thereof is disclosed. The source or cause of the disease may for example be a virus, a bacteria, a fungi, and/or parasites.

The system is configured to obtain, e.g., via the electronic device (such as using the processor and the interface of the electronic device) and/or the server device (such as using the one or more processors and the interface of the server device), a hospitalization status of a patient. The patient may be a person who has been tested positive for a disease, such as the disease to be monitored/handled. The patient may be a person being in a high risk category with respect to the disease to be monitored/handled. The patient may be a potential patient to be hospitalized. In one or more exemplary systems, the hospitalization status may be indicative of and/or comprise information on whether the patient is hospitalized or not. If the patient is hospitalized, the hospitalization status May comprise information on whether the patient is home hospitalized or hospitalized in a hospital. The hospitalization status may be obtained from a social registry database, such as social health registry database, via a network such as a global network, e.g. the internet.

In one or more exemplary systems, the hospitalization status of the patient comprises information on whether the patient is hospitalized or not.

In one or more exemplary systems, when the patient is hospitalized the hospitalization status of the patient comprises information on whether the patient is hospitalized in a hospital or home hospitalized. Home hospitalized may for example be seen as an actual home of a patient, a nursing home, and/or an assisted housing.

In one or more exemplary systems, the hospitalization status may be used to determine whether a discharge recommendation of the patient may be determined. In other words, the system may determine a discharge recommendation based on the hospitalization status. For example, when the hospitalization status indicates that the patient is hospitalized in a hospital, the system may be configured to determine a discharge recommendation indicating that a patient can be discharged from the hospital and instead be home hospitalized. For example, when the hospitalization status indicates that the patient is home hospitalized, the system may determine a discharge recommendation indicating that a patient can be discharged from a home hospitalization and stop being monitored at home.

The system is configured to obtain patient data, also denoted PD, of the patient, e.g., using the processor and/or the interface of the electronic device. In one or more exemplary systems, to obtain patient data PD, comprises to obtain first patient data, also denoted PD_1.

In one or more exemplary systems, the first patient data PD_1 comprises patient status data, also denoted PSD, and symptom data, also denoted SD. In one or more exemplary systems, the first patient data PD_1 comprises first patient status data, also denoted PSD_1, and first symptom data, also denoted SD_1. The first patient status data PSD_1 may be indicative of a first status of the patient after a first screening and/or a first consultation, such as a video consultation. The first symptom data SD_1 may be indicative of first symptoms of the patient after the first screening and/or the first consultation. The first patient data PD_1 may be obtained via an interface of the electronic device, e.g. a user interface of the electronic device. To obtain the first patient data may comprise to perform a first screening and/or a first consultation of the patient. In other words, to obtain the first patient data may comprise to perform a first iteration of a screening and/or a first iteration of a consultation of the patient, such as a first iteration of a video consultation. The first screening and/or first consultation may be performed via the electronic device, such as via the user interface of the electronic device. The first screening may be seen as a pre-screening of the patient, e.g, for obtaining pre-screening data of the patient. A pre-screening may be conducted to determine whether a patient is suitable or not to be hospitalized, such as home hospitalized. The first patient data may be based on a first user/patient input. The first patient data may comprise one or more user/patient inputs, including a first patient input. For example, the first patient data, such as the outcome of the first screening and/or the first consultation, may comprise to obtain the first patient input comprising the patient manually inputting patient data such as patient status data and symptom data.

The first patient data may comprise static patient data, e.g. provided by the patient as first primary patient input. The first patient data may comprise patient identification data, such as social register number, a personal identification number, a social security number, and/or a social health security number. For example, the first patient data may comprise obtaining the first patient input comprising the patient manually inputting patient data such as age, gender, height, weight, health condition/health data, and/or area of residence (such as a municipality). The first patient data may comprise a first timestamp. The first timestamp may at least be indicative of a day. The first timestamp may be indicative of a day and time of day. The first patient data may comprise patient status data such as pre-screening data associated with a patient. For example, to obtain first patient data may comprise to obtain first patient data via the first screening comprising a pre-screening of the patient. The pre-screening data may be used to determine whether a patient is suitable or not to be hospitalized, such as home hospitalized. To obtain first patient data may comprise to obtain consent data from a patient, such as a written or oral consent to be home hospitalized.

In one or more exemplary systems, the system is configured to determine whether the first patient data satisfies an inclusion criterion, such as satisfies one or more inclusion criteria. The inclusion criterion may be seen as or comprise a criterion or criteria indicating a patient's suitability to be hospitalized, such as home hospitalized. In other words, when it is determined that the first patient data satisfies the inclusion criterion, it may be determined that the patient is suitable for home hospitalization. The inclusion criterion may comprise an age criterion, such as an age threshold. For example, the first patient data satisfies the inclusion criterion when the age of the patient is above or equal to 18 years old.

The inclusion criterion may comprise a consent criterion. The system may be configured to determine whether the first patient data satisfies the consent criterion. For example, the first patient data satisfies the inclusion criterion when the patient has given consent to be home hospitalized.

The first patient data may comprise information regarding a pre-condition of a patient, such as a pre-contracted disease. The inclusion criterion may comprise a disease criterion. The system may be configured to determine whether the first patient data satisfies the disease criterion. For example, the first patient data satisfies the inclusion criterion when the patient has contracted a respiratory infection, such as a lower tract respiratory infection, e.g., a contagious infection, such as SARS COV-2 or influenza.

The first patient data may comprise care information indicating whether a patient receives formal care and/or informal care. The inclusion criterion may comprise an independency criterion. The system may be configured to determine whether the first patient data satisfies the independency criterion. For example, the first patient data satisfies the inclusion criterion when the patient is sufficiently independent (such as self-reliant), e.g., formal care-independent, to be home hospitalized. For example, when a patient receives sufficient informal care from an informal caregiver, the patient may be formal care independent.

The inclusion criterion may comprise a geographical area criterion. The system may be configured to determine whether the first patient data satisfies the geographical area criterion. For example, the first patient data satisfies the inclusion criterion when the patient's address is within a certain geographical area from a hospital. In other words, the first patient data may satisfy the inclusion criterion when the patient's address is located within a catchment area of a hospital.

The inclusion criterion may comprise a medical eligibility criterion, such as medical eligibility criterion for home hospitalization. The system may be configured to determine whether the first patient data satisfies the medical eligibility criterion. For example, the first patient data satisfies the inclusion criterion when the patient's medical condition satisfies one or more medical eligibility criteria. For example, the medical eligibility criterion may comprise one or more of: a health condition criterion, an early warning score criterion, an oxygen supplement criterion, and an infection parameter criterion. The system may be configured to determine whether the first patient data satisfies the health condition criterion. For example, the first patient data satisfies the inclusion criterion, such as health condition criterion, when the patient's health condition is clinically stable. The system may be configured to determine whether the first patient data satisfies the early warning score criterion. For example, the first patient data satisfies the inclusion criterion, such early warning score criterion, when the patient's early warning score is below or equal to 6 and that no single parameter score of the early warning score is equal to or above 3. The system may be configured to determine whether the first patient data satisfies the oxygen supplement criterion. For example, the first patient data satisfies the inclusion criterion, such as oxygen supplement criterion, when the patient requires less than 5 L/min of oxygen supplement and/or that the need for oxygen supplement of the patient is stable or decreasing. The system may be configured to determine whether the first patient data satisfies the infection parameter criterion. For example, the first patient data satisfies the inclusion criterion, such as infection parameter criterion, when the patient's infection parameters, such as paraclinical infection parameters, e.g., C-reactive protein, are stable or decreasing.

In one or more exemplary systems, the system is configured to determine whether the first patient data satisfies an exclusion criterion, such as satisfies one or more exclusion criteria. The exclusion criterion may be seen as or comprise a criterion or a plurality of criteria indicating a patient's unsuitability to be hospitalized, such as home hospitalized. When it is determined that the first patient data satisfies the exclusion criterion, it may be determined that the patient is unsuitable for home hospitalization.

The exclusion criterion may comprise a digital competency criterion indicative of a digital competence of a patient. The system may be configured to determine whether the first patient data satisfies the digital competency criterion. For example, the first patient data satisfies the exclusion criterion when the patient is determined to be digitally incompetent.

The exclusion criterion may comprise a language criterion indicative of a language competence of a patient, such as language proficiency of a patient. The system may be configured to determine whether the first patient data satisfies the language criterion. For example, the first patient data satisfies the exclusion criterion when the patient is determined to have a language competence and/or language proficiency below a language competence threshold.

The exclusion criterion may comprise an isolation criterion indicative of a possibility of isolation of a patient at his premises. The system may be configured to determine whether the first patient data satisfies the isolation criterion. For example, the first patient data satisfies the exclusion criterion when it is determined that the patient cannot be in isolation at his premises.

The exclusion criterion may comprise a pregnancy criterion indicative of a pregnancy state of a patient. The system may be configured to determine whether the first patient data satisfies the pregnancy criterion. For example, the first patient data satisfies the exclusion criterion when the patient is pregnant.

The exclusion criterion may comprise a diabetes criterion indicative of a diabetes condition of a patient. The system may be configured to determine whether the first patient data satisfies the diabetes criterion. For example, the first patient data satisfies the exclusion criterion when the patient has unstable diabetes, such as unstable type 1 or type 2 diabetes. For example, the first patient data satisfies the exclusion criterion when the patient is not used to take insulin.

In one or more exemplary systems, the patient status data is indicative of one or more of temperature, pulse, respiratory rate, oxygen saturation, consciousness, blood sugar, and blood pressure of the patient. For example, the patient status data may comprise to obtain the first patient input comprising the patient manually inputting patient status data such as one or more of temperature, pulse, respiratory rate, oxygen saturation, consciousness, blood sugar, and blood pressure. The patient may for example receive instructions from a health care professional (such as a caretaker) to measure one or more of his/her temperature, pulse, respiratory rate, oxygen saturation, and blood pressure. The patient may for example have received equipment to measure one or more of his/her temperature, pulse, respiratory rate, oxygen saturation, and blood pressure. The patient may additionally or alternatively have equipment themselves to measure patient status data such as one or more parameter, e.g. one or more of temperature, pulse, respiratory rate, oxygen saturation, and blood pressure.

The symptom data is indicative of one or more symptoms of the patient. For example, the symptom data may comprise to obtain the first patient input comprising the patient manually inputting symptom data such as one or more of symptoms felt by the patient, e.g. via the interface of the electronic device. The symptom data may for example comprise one or more of coughing, having a sore throat, dizziness, stomach pain, diarrhea, nausea, shortness of breath, respiratory problems, headache, vomiting, confusion, tired, tasting and/or smelling issues.

The symptom data may comprise information about whether the patient have had a sore throat (such as swallowing pain) in the past day(s) (e.g. past 24 hours). The symptom data may comprise information about whether the patient have had fever in the past day(s). The symptom data may comprise information about whether the patient have had muscle pain or soreness in the past day(s). The symptom data may comprise information about whether the patient have had shortness of breath in the past day(s). The symptom data may comprise information about whether the patient have had respiratory problems in the past day(s). The symptom data may comprise information about whether the patient have had a headache in the past day(s). The symptom data may comprise information about whether the patient have had dizziness in the past day(s). The symptom data may comprise information about whether the patient have had stomach pain in the past day(s). The symptom data may comprise information about whether the patient have had diarrhea in the past day(s). The symptom data may comprise information about whether the patient have had nausea in the past day(s). The symptom data may comprise information about whether the patient has been vomiting in the past day(s). The symptom data may comprise information about whether the patient has been more confused than normal in the past day(s). The symptom data may comprise information about whether the patient has been more tired than normal in the past day(s). The symptom data may comprise information about whether the patient has been coughing more than normal in the past day(s). The symptom data may comprise information about whether the patient have had more difficulties tasting or smelling than normal in the past day(s).

The system is configured to determine a first patient metric, also denoted PM_1, based on the first patient data. For example, the system may be configured to determine the first patient metric using the electronic device (such as using the processor and/or the interface of the electronic device) and/or using the server device (such as using the one or more processors and/or the interface of the server device). In one or more exemplary systems, to determine a first patient metric may comprise to determine a first patient metric based on the patient status data and/or the symptom data. The first patient metric PM_1 may be determined based on an algorithm. In one or more exemplary systems, the system is configured to determine the first patient metric based on the first patient data and the hospitalization status.

The first patient metric PM_1 may be indicative of a condition of a patient, such as a health condition of a patient.

The first patient metric may be determined based on one or more parameters comprised in the first patient data. In other words, the first patient metric may be determined based on the first user input. The first patient metric may comprise a weighted score. For example, the first patient metric may be weighted based on a weighing of the obtained first patient data, e.g. on a weighing of the patient status data and/or symptom data, such as inputted by the patient. For example, certain symptoms may have a larger weighting coefficient than others, e.g. depending on the disease to be monitored and/or handled. The weighting of the first patient data may depend on the disease that is to be monitored.

The first patient metric PM_1 may comprise a score based on the first patient data.

The first patient data PD_1 may comprise one or more parameters indicative of a patient status and/or a symptom of a patient. The one or more parameters may for example be based on one or more answers from a patient when performing a screening (such as a questionnaire). The system may be configured to determine a first patient metric PM_1 based on the one or more parameter, such as based on a first parameter, a second parameter, a third parameter, a fourth parameter, a fifth parameter, sixth parameter, etc. The one or more parameters may be obtained when a patient performs a screening and/or a consultation (such as the first screening and/or the second screening). For example, the system may be configured to assign and/or determine a value and/or a score to/of the first patient metric PM_1 and/or the second patient metric PM_2 based on the first patient data PD_1. The value and/or score may e.g. be in the range of 0 to 3, such as 0, 0.5, 1, 1.5, 2, 2.5, or 3. The value or score 0 may be indicative of the lowest value or score and the value or score 3 may be indicative of the highest value or score. The value or score 0 may be indicative of a “normal” condition, such as the patient having no symptoms. The value or score 3 may be indicative of an “abnormal” condition, such as the patient having critical or severe symptoms. The value or score 1 may be indicative of an “abnormal” condition, such as the patient having mild symptoms. The value or score 2 may be indicative of an “abnormal” condition, such as the patient having moderate to severe symptoms. A screening as disclosed herein may be seen as a questionnaire.

For example, the first patient data, such as the patient status data, may comprise a first parameter indicative of a respiratory rate of a patient. The first parameter may be indicative of different ranges of respiratory rates, such as above 24 breaths per minute, in the range of 21-24 breaths per minute, 12-20 breaths per minute, 9-11 breaths per minute, or below 9 breaths per minute. For example, the first parameter (such as a respiratory rate parameter) may be indicative of a respiratory rate of above 24 breaths per minute. When the first parameter is indicative of a respiratory rate of above 24 breaths per minute, the system may be configured to assign (such as determine) the first patient metric PM_1 with the value or score 3. When the first parameter is indicative of a respiratory rate in the range of 21-24 breaths per minute, the system may be configured to assign the first patient metric PM_1 with the value or score 2. When the first parameter is indicative of a respiratory rate in the range of 12-20 breaths per minute, the system may be configured to assign the first patient metric PM_1 with the value or score 0. When the first parameter is indicative of a respiratory rate in the range of 9-11 breaths per minute, the system may be configured to assign the first patient metric PM_1 with the value or score 1. When the first parameter is indicative of a respiratory rate of below 9 breaths per minute, the system may be configured to assign the first patient metric PM_1 with the value or score 3.

For example, the first patient data, such as the patient status data, may comprise a second parameter indicative of an oxygen saturation of a patient (such as oxygen saturation percentage). The second parameter may be indicative of different ranges of oxygen saturation percentages, such as above 95% of oxygen saturation, in the range of 94-95%, 92-93%, or below 92%. For example, the second parameter (such as an oxygen saturation parameter) may be indicative of an oxygen saturation percentage of above 95%. When the second parameter is indicative of an oxygen saturation percentage of above 95%, the system may be configured to assign the first patient metric PM_1 with the value or score 0. When the second parameter is indicative of an oxygen saturation percentage in the range of 94-95%, the system may be configured to assign (such as determine) the first patient metric PM_1 with the value or score 1. When the second parameter is indicative of an oxygen saturation percentage in the range of 92-93, the system may be configured to assign the first patient metric PM_1 with the value or score 2. When the second parameter is indicative of an oxygen saturation percentage below 92%, the system may be configured to assign the first patient metric PM_1 with the value or score 3. When the patient receives oxygen therapy (such as oxygen supplement), the system may be configured to assign the first patient metric PM_1 with the value or score 2.

For example, the first patient data, such as the patient status data, may comprise a third parameter indicative of a pulse of a patient. The third parameter may be indicative of different ranges of pulses per minute, such as above 130 pulses per minute, in the range of 111-130 pulses per minute, 91-110 pulses per minute, 51-90 pulses per minute, 41-50 pulses per minute, or below 41 pulses per minute. When the third parameter is indicative of a pulse per minute of above 130 pulses per minute, the system may be configured to assign (such as determine) the first patient metric PM_1 with the value or score 3. When the third parameter is indicative of a pulse per minute in the range of 111-130, the system may be configured to assign the first patient metric PM_1 with the value or score 2. When the third parameter is indicative of a pulse per minute in the range of 91-110, the system may be configured to assign the first patient metric PM_1 with the value or score 1. When the third parameter is indicative of a pulse per minute in the range of 51-90 pulses per minute, the system may be configured to assign the first patient metric PM_1 with the value or score 0. When the third parameter is indicative of a pulse per minute in the range of 41-50 pulses per minute, the system may be configured to assign the first patient metric PM_1 with the value or score 1. When the third parameter is indicative of a pulses per minute of below 41 pulses per minute, the system may be configured to assign the first patient metric PM_1 with the value or score 3.

For example, the first patient data, such as the patient status data, may comprise a fourth parameter indicative of a blood pressure of a patient (such as a systolic blood pressure). The fourth parameter may be indicative of different ranges of blood pressure per mmHg, such as above 219 mmHg, in the range of 111-219 mmHg, 101-110 mmHg, 91-100 mmHg, or below 91 mmHg. When the fourth parameter is indicative of a blood pressure of above 219 mmHg, the system may be configured to assign the first patient metric PM_1 with the value or score 3. When the fourth parameter is indicative of a blood pressure in the range of 111-219 mmHg, the system may be configured to assign the first patient metric PM_1 with the value or score 0. When the fourth parameter is indicative of a blood pressure in the range of 101-110 mmHg, the system may be configured to assign the first patient metric PM_1 with the value or score 1. When the fourth parameter is indicative of a blood pressure in the range of 91-100 mmHg, the system may be configured to assign the first patient metric PM_1 with the value or score 2. When the fourth parameter is indicative of a blood pressure of below 91 mmHg, the system may be configured to assign the first patient metric PM_1 with the value or score 3.

For example, the first patient data, such as the patient status data, may comprise a fifth parameter indicative of a consciousness of a patient (such as alertness and/or vigilance of a patient). The fifth parameter may be indicative of a state of consciousness of a patient, such as normal consciousness or reduced consciousness. In other words, the fifth parameter may be indicative of a state of the sleeping of a patient, such as normal sleep or increased sleep (such as increased need for sleep, e.g., increased sleepiness). When the fifth parameter is indicative of a normal consciousness, the system may be configured to assign the first patient metric PM_1 with the value or score 0. When the fifth parameter is indicative of a reduced consciousness, the system may be configured to assign the first patient metric PM_1 with the value or score 3. For example, when a patient is able to complete a screening (such as the first screening), the system may be configured assign the first patient metric PM_1 with the value or score 0.

For example, the first patient data, such as the patient status data, may comprise a sixth parameter indicative of a temperature of a patient (such as a body temperature of a patient). The sixth parameter may be indicative of different ranges of temperatures, such as above 39.0° C., in the range of 38.1° C.-39.0° C., 36.1° C.-38.0° C., 35.1° C.-36.0° C., or below 35.1° C. When the sixth parameter is indicative of a temperature of above 39.0° C., the system may be configured to assign the first patient metric PM_1 with the value or score 2. When the sixth parameter is indicative of a temperature in the range of 38.1° C.-39.0° C., the system may be configured to assign the first patient metric PM_1 with the value or score 1. When the sixth parameter is indicative of a temperature in the range of 36.1° C.-38.0° C., the system may be configured to assign the first patient metric PM_1 with the value or score 0. When the sixth parameter is indicative of a temperature in the range of 35.1° C.-36.0° ° C., the system may be configured to assign the first patient metric PM_1 with the value or score 1. When the sixth parameter is indicative of a temperature of below 35.1° C., the system may be configured to assign the first patient metric PM_1 with the value or score 3.

The description of the first patient metric PM_1 may also apply to the second patient metric PM_2 such as the description of the determination of the first patient metric PM_1 may also apply to the second patient metric PM_2. The description of the first patient data PD_1 may also apply to the second patient data PD_2.

The system is configured to determine whether the first patient metric PM_1 satisfies a first criterion, also denoted C_1, e.g., using the electronic device (such as using the processor and/or the interface of the electronic device) and/or using the server device (such as using the one or more processors and/or the interface of the server device).

In other words, the system is configured to determine whether the first patient metric PM_1 satisfies or fulfills the first criterion C_1.

The first criterion may be indicative of a threshold value, such as a first threshold value, that the first patient metric satisfies or not. The first criterion may be indicative of whether the patient shall be hospitalized or not. The first criterion may be indicative of whether the patient shall be hospitalized in a hospital or home hospitalized.

In one or more exemplary systems, to determine whether the first patient metric PM_1 satisfies a first criterion C_1 comprises to determine whether the first patient metric is above a first threshold or within a first range of the first criterion.

In one or more exemplary systems, to determine whether the first patient metric PM_1 satisfies a first criterion C_1 comprises to determine whether the first patient metric is indicative of (such as corresponds to) the patient having mild symptoms, no symptoms, critical or severe symptoms. In other words, when the first patient metric PM_1 satisfies the first criterion C_1 the first patient metric PM_1 may be indicative of the patient having at least mild symptoms, such as the patient having at least intermediate symptoms.

In one or more exemplary systems, to determine whether the first patient metric PM_1 satisfies a first criterion C_1 comprises to determine whether the first patient metric is indicative of (such as corresponds to) an abnormal condition of the patient.

The system is configured in accordance with the first patient metric PM_1 satisfying the first criterion C_1, to output a first action request, e.g. via the electronic device (such as via the interface of the electronic device). In one or more exemplary systems, the system is configured, in accordance with the first patient metric PM_1 satisfying the first criterion C_1 and based on the hospitalization status of the patient, to output a first action request, e.g. via the electronic device (such as via the interface of the electronic device).

In one or more exemplary systems, to output a first action request comprises to determine the first action request based on the determination of whether the first patient metric satisfies the first criterion.

In other words, the system may be configured to determine whether an action request, such as a first action request, e.g. an advisory action request, should be taken, in view of the determined first patient metric. For example, when the first patient metric satisfies the first criterion, there may be a probability that the patient has the disease, has severe symptoms related to the disease, and/or that the patient is classified as being in a vulnerable group. The first action request may comprise a minimum observation interval of the patient, such as to output a minimum observation interval of the patient. In other words, the system may be configured to determine a minimum observation interval of the patient based on the first patient metric. In one or more exemplary systems, to output a first action request comprises to determine the first action request based on the determination of whether the first patient metric satisfies the first criterion and the hospitalization status. In other words, the system may be configured to determine whether an action request, such as a first action request, e.g. an advisory action request, should be taken, in view of the determined first patient metric and the hospitalization status of the patient.

For example, when the first patient metric is equal to the first threshold, it may still be determined that the first patient metric satisfies the first criterion for precautionary reasons. The first action request may comprise to display a message or information text, e.g. via the electronic device, to the patient. The message or information text may be indicative of a first action request that the patient may react on or take action on. For example, the first action request may comprise to indicate to the patient to perform an additional screening and/or a consultation in order to clarify whether the patient is in danger and/or should be hospitalized or not. The patient may for example perform an additional screening and/or a consultation after a certain period of time after the first screening and/or consultation has been performed. The first action request may comprise to indicate to the patient that he/she is in an exposed category with regard to a disease and should take precautionary measures, e.g. how to take precautionary measures and/or what actions to perform. For example, try to avoid being in contact with anyone and, if to be hospitalized, the course of action for the hospitalization (at home or at a hospital). The first action request may provide guidance to patients in relation to how to handle their condition, symptoms, and disease in general e.g. when a pandemic or epidemic are present.

In one or more exemplary system, to output a first action request comprises to display, e.g. on the interface of the electronic device, a first user interface, indicative of the first action request and/or indicative of, e.g. being, a link to a disease monitoring/handling program or website. A first user interface may be understood as a first patient interface.

A user interface may comprise one or more, such as a plurality of user interface objects. For example, the first user interface may comprise one or more first user interface objects, such as a first primary user interface object and/or a first secondary user interface object. The first user interface may comprise to display the first action request, such as a message or information text, e.g. via the electronic device, to the patient. The message or information text may be indicative of a first action request that the patient may react on or take action on.

The first action request may be comprised in a first user interface object, such as a first primary user interface object. For example, a first action request may include a message such as: “We recommend that you contact your doctor”. For example, a first action request may include a message such as: “We recommend that you stay at home and avoid as much as possible to get in contact with other people outside from your household”. For example, a first action request may include a message such as: “We recommend that you get home hospitalized”. For example, a first action request may include a message such as: “We recommend that you get hospitalized in a hospital”. For example, a first action request may include a message such as: “We recommend activating a virtual epidemic centre/virtual monitoring centre and you will get contacted by telephone”. For example, a first action request may include a message such as: “We recommend that you perform an additional screening and/or a consultation with a health care professional”. For example, a first action request may include a message such as: “We recommend that you perform a video consultation with a health care professional”.

In one or more example systems, the system is configured to detect a selection of the first user interface object, e.g. the user selecting or clicking in the disease monitoring/handling program or website. A user interface object may refer herein to a graphical representation of an object that is displayed on a display of the electronic device. The user interface object may be user-interactive, or selectable by a user input. For example, an image (e.g., icon), a button, and text (e.g., hyperlink) each optionally constituting a user interface object. The user interface object may form part of a widget. A widget may be seen as a mini-application that may be used by the user. The user interface object may allow the patient to input first patient data, such as patient status data and symptom data.

In one or more exemplary systems, when the first patient metric does not satisfy the first criterion, refrain from outputting a first action request, such as “We currently do not recommend a hospitalization”. In one or more exemplary systems, when the first patient metric does not satisfy the first criterion, refrain from outputting a first action request. In one or more exemplary systems, the first patient metric PM_1 does not satisfy the first criterion C_1 when the first patient metric PM_1 is equal to or below a first threshold or within a second range of the first criterion.

In one or more exemplary systems, the system is configured to obtain second patient data, also denoted PD_2, of the patient, e.g. via the electronic device (such as using the processor and the interface of the electronic device) and/or the server device (such as using the one or more processors and the interface of the server device).

In one or more exemplary systems, the second patient data PD_2 comprises patient status data, also denoted PSD, and symptom data, also denoted SD. In one or more exemplary systems, the second patient data PD_2 comprises second patient status data, also denoted PSD_2, and second symptom data, also denoted SD_2. The second patient status data PSD_2 may be indicative of a status of the patient after a second screening and/or a second consultation, such as a second video consultation. The second symptom data SD_2 may be indicative of second symptoms of the patient after the second screening and/or the second consultation. In other words, the second patient data PD_2 may comprise updated patient data, such as updated patient status data and/or updated symptom data. The second patient data PD_2 may be different from the first patient data PD_1. For example, the second patient data PD_2 may indicate that the patient status data PSD and/or the symptom data SD of the patient have changed. The second patient data PD_2 may for example comprise second patient status data PSD_2 indicating that the temperature of the patient is now 39° C. and has increased by 1° C. from the first patient status data.

The second patient data PD_2 may in some cases be the same as the first patient data PD_1, when the patient data is unchanged.

The second patient data PD_2 may be obtained via an interface of the electronic device, e.g. a user interface of the electronic device. To obtain the second patient data may comprise to perform a second screening and/or a second consultation of the patient. In other words, to obtain the second patient data may comprise to perform a second iteration of a screening and/or a second iteration of a consultation of the patient. The second screening and/or second consultation may be performed via the electronic device, such as via the user interface of the electronic device. The second patient data may be based on a second user/patient input. The second patient data may comprise one or more user/patient inputs, including a second patient input. For example, the second patient data, such as the second screening and/or the second consultation, may comprise to obtain the second patient input comprising the patient manually inputting patient data such as patient status data and symptom data.

The second patient data may comprise static patient data, e.g. provided by the patient as second primary patient input. The second patient data may comprise patient identification data, such as social register number, a personal identification number, a social security number, and/or a social health security number. For example, the second patient data may comprise obtaining the second patient input comprising the patient manually inputting patient data such as age, gender, height, weight, health condition/health data, and/or area of residence (such as a municipality). The second patient data may comprise a second timestamp. The second timestamp may at least be indicative of a day. The second timestamp may be indicative of a day and time of day.

In one or more exemplary systems, the patient status data PSD, such as the second patient status data PSD_2, is indicative of one or more of temperature, pulse, respiratory rate, oxygen saturation, and blood pressure of the patient.

For example, the patient status data, such as second patient status data, may comprise to obtain the second patient input comprising the patient manually inputting patient status data, e.g. second patient status data, such as one or more of temperature, pulse, respiratory rate, oxygen saturation, and blood pressure. The patient may for example receive instructions from a health care professional to measure one or more of his/her temperature, pulse, respiratory rate, oxygen saturation, and blood pressure during the second screening and/or consultation. The patient may for example have received equipment to measure one or more of his/her temperature, pulse, respiratory rate, oxygen saturation, and blood pressure. The patient may additionally or alternatively have equipment themselves to measure patient status data such as one or more parameter, e.g. one or more of temperature, pulse, respiratory rate, oxygen saturation, and blood pressure.

In one or more exemplary systems, the symptom data being indicative of one or more symptoms of the patient. For example, the symptom data may comprise to obtain the second patient input comprising the patient manually inputting symptom data, such as second symptom data, such as one or more of symptoms felt by the patient, e.g. via the interface of the electronic device. The symptom data, such as second symptom data, may for example comprise one or more of coughing, having a sore throat, dizziness, stomach pain, diarrhea, nausea, shortness of breath, respiratory problems, headache, vomiting, confusion, tired, tasting and/or smelling issues.

In one or more exemplary systems, the system is configured to determine a second patient metric, also denoted PM_2, based on the second patient data. In one or more exemplary systems, to determine a second patient metric may comprise to determine a second patient metric based on the patient status data and/or the symptom data. In one or more exemplary systems, to determine a second patient metric may comprise to determine a second patient metric based on the first patient status data, the second patient status data, the first symptom data, and/or the second symptom data. The second patient metric PM_2 may be determined based on an algorithm. In one or more exemplary systems, the system is configured to determine the second patient metric based on the second patient data and the hospitalization status.

The second patient metric PM_2 may be indicative of a condition of a patient, such as a health condition of a patient.

The second patient metric may be determined based on one or more parameters comprised in the second patient data. In other words, the second patient metric may be determined based on the second user input. The second patient metric may comprise a weighted score. For example, the second patient metric may be weighted based on a weighing of the obtained second patient data, e.g. on a weighing of the patient status data and/or symptom data, such as inputted by the patient. For example, certain symptoms may have a larger weighting coefficient than others, e.g. depending on the disease to be monitored and/or handled. The weighting of the second patient data may depend on the disease that is to be monitored.

The second patient metric PM_1 may comprise a score based on the second patient data.

In one or more exemplary systems, the system is configured to determine whether the second patient metric PM_2 satisfies the first criterion C_1.

In other words, the system is configured to determine whether the second patient metric PM_1 satisfies or fulfills the first criterion C_1.

The first criterion may be indicative of a threshold value, such as a first threshold value, that the second patient metric satisfies or not. The first criterion may be indicative of whether the patient shall be hospitalized or not. The first criterion may be indicative of whether the patient shall be hospitalized in a hospital or home hospitalized.

In one or more exemplary systems, to determine whether the second patient metric PM_2 satisfies a first criterion C_1 comprises to determine whether the second patient metric is above a first threshold or within a first range of the first criterion.

In one or more exemplary systems, to determine whether the second patient metric PM_2 satisfies a first criterion C_1 comprises to determine whether the second patient metric corresponds to mild symptoms, no symptoms, or critical symptoms.

In one or more exemplary systems, the system is configured in accordance with the second patient metric PM_2 satisfying the first criterion C_1, to output a second action request.

In one or more exemplary systems, to output a second action request comprises to determine the second action request based on the determination of whether the second patient metric satisfies the first criterion. In one or more exemplary systems, to output a second action request comprises to determine the second action request based on the determination of whether the second patient metric satisfies the second criterion and the hospitalization status. In other words, the system may be configured to determine whether an action request, such as a second action request, e.g. an advisory action request, should be taken, in view of the determined second patient metric and the hospitalization status of the patient.

In other words, the system may be configured to determine whether an action request, such as a second action request, e.g. an advisory action request, should be taken in view of the determined second patient metric. For example, when the second patient metric satisfies the first criterion, there may be a probability that the patient has the disease, has severe symptoms related to the disease, and/or that the patient is classified as being in a vulnerable group.

For example, when the second patient metric is equal to the first threshold, it may still be determined that the second patient metric satisfies the first criterion for precautionary reasons. The second action request may comprise to display a message or information text, e.g. via the electronic device, to the patient. The message or information text may be indicative of a second action request that the patient may react on or take action on. The second action request should be taken in view of the second patient metric compared to the first patient metric. The second action request may comprise to indicate to the patient that his/her condition has improved or deteriorated.

For example, the second action request may comprise to indicate to the patient to perform an additional screening and/or a consultation in order to clarify whether the patient is in danger and/or should be hospitalized or not. The patient may for example perform an additional screening and/or a consultation after a certain period of time after the second screening and/or consultation has been performed. The second action request may comprise to indicate to the patient that he/she is in an exposed category with regard to a disease and should take precautionary measures, e.g. how to take precautionary measures and/or what actions to perform. For example, try to avoid being in contact with anyone and, if to be hospitalized, the course of action for the hospitalization (at home or at a hospital). The second action request may provide guidance to patients in relation to how to handle their condition, symptoms, and disease in general e.g. when a pandemic or epidemic are present.

In one or more exemplary system, to output a second action request comprises to display, e.g. on the interface of the electronic device, a second user interface, indicative of the second action request and/or indicative of, e.g. being, a link to a disease monitoring/handling program or website. A second user interface may be understood as a second patient interface.

A user interface may comprise one or more, such as a plurality of user interface objects. For example, the second user interface may comprise one or more second user interface objects, such as a second primary user interface object and/or a second secondary user interface object. The second user interface may comprise to display the second action request, such as a message or information text, e.g. via the electronic device, to the patient. The message or information text may be indicative of a second action request that the patient may react on or take action on.

The second action request may be comprised in a second user interface object, such as a second primary user interface object. For example, a second action request may include a message such as: “We recommend that you contact your doctor”. For example, a second action request may include a message such as: “We recommend that you stay at home and avoid as much as possible to get in contact with other people outside from your household”. For example, a second action request may include a message such as: “We recommend that you get home hospitalized”. For example, a second action request may include a message such as: “We recommend that you get hospitalized in a hospital”. For example, a second action request may include a message such as: “We recommend activating a virtual epidemic centre and you will get contacted by telephone”. For example, a second action request may include a message such as: “We recommend that you perform an additional screening and/or a consultation with a health care professional”.

In one or more example systems, the system is configured to detect a selection of the second user interface object, e.g. the user selecting or clicking in the disease monitoring/handling program or website. A user interface object may refer herein to a graphical representation of an object that is displayed on a display of the electronic device. The user interface object may be user-interactive, or selectable by a user input. For example, an image (e.g., icon), a button, and text (e.g., hyperlink) each optionally constituting a user interface object. The user interface object may form part of a widget. A widget may be seen as a mini-application that may be used by the user. The user interface object may allow the patient to input second patient data, such as patient status data and symptom data.

In one or more exemplary systems, when the second patient metric does not satisfy the first criterion, refrain from outputting a second action request, such as “We currently do not recommend a hospitalization”. In one or more exemplary systems, when the second patient metric does not satisfy the first criterion, refrain from outputting a second action request, i.e. do not output a second action request.

In one or more exemplary systems, the first action request and/or the second action request comprises one or more of a request to contact a doctor, a request to activate a virtual monitoring center, a request for home hospitalization, and a request for hospitalization in a hospital.

In one or more exemplary systems, when the hospitalization status indicates that a patient is not hospitalized, a request for home hospitalization may indicate that a patient should go from not hospitalized to home hospitalized.

In one or more exemplary systems, when the hospitalization status indicates that a patient is hospitalized in a hospital, a request for home hospitalization may indicate that a patient does not need inpatient care in a hospital and can be transferred from hospitalized to home hospitalized.

In one or more exemplary systems, when the hospitalization status indicates that a patient is home hospitalized, a request for hospitalization in a hospital may indicate that a patient should go from home hospitalized to hospitalized in a hospital.

In one or more exemplary systems, when the hospitalization status indicates that a patient is home hospitalized, the first action request and/or the second action request may comprise a request to activate a virtual monitoring center.

In one or more exemplary systems, the system is configured to determine a patient data scheme based on the first patient metric, e.g. using the electronic device (such as using the processor and the interface of the electronic device) and/or the server device (such as using the one or more processors and the interface of the server device, for example receiving the patient data scheme from the server device at the electronic device). The patient data scheme may comprise or define a frequency to obtain patient data, such as first patient data and/or second patient data. The patient data scheme may comprise or define a schedule to obtain patient data, such as first patient data and/or second patient data. In other words, the patient data scheme may comprise or define one or more iterations or cycles of screenings and/or consultations of the patient.

In one or more exemplary systems, to obtain the second patient data is based on the patient data scheme. In other words, the patient data scheme may comprise or define a point in time, such as a date and/or a time, where the second patient data shall be obtained. In other words, the patient data scheme may comprise or define a point in time where the second screening and/or the second consultation is to be performed by the patient.

In one or more exemplary systems, the patient data scheme comprises a first time parameter indicative of a time between the obtaining of the first patient data and the obtaining of the second patient data. The first time parameter may comprise or define a point in time where the second screening and/or the second consultation is to be performed by the patient. In other words, first time parameter may comprise or define a screening frequency of the patient. For example, the first time parameter may be indicative of a time between the obtaining of the first patient data and the obtaining of the second patient data of at least 2 hours, at least 4 hours, or at least 8 hours. In one or more exemplary systems, the second patient data may be obtained every 2 hours, every 4 hours, or every 8 hour.

The first time parameter may depend on the first patient data, such as the patient status data and the symptom data, and/or the first patient metric. The first time parameter may depend on whether the first patient metric satisfies the first criterion. For example, when the first patient metric satisfies the first criterion, the first time parameter may indicate a shorter time between the obtaining of the first patient data and the obtaining of the second patient data.

In one or more exemplary systems, to obtain second patient data PD_2 comprises to determine whether a patient data criterion, also denoted PDC, based on the first time parameter is satisfied, e.g. using the electronic device (such as using the processor and the interface of the electronic device) and/or the server device (such as using the one or more processors and the interface of the server device). In other words, the system is configured to determine whether the second patient data PD_2 satisfies or fulfills the patient data criterion PDC based on the first time parameter. The system may be configured to determine whether the patient status data and/or the symptom data have changed in the time between the obtaining of the first patient data and the obtaining of the second patient data.

The patient data criterion may be indicative of a threshold value, such as a patient data threshold value, that the second patient data satisfies or not.

The patient data criterion may be indicative of whether the patient shall be hospitalized or not. The patient data criterion may be indicative of whether the patient shall be hospitalized in a hospital or home hospitalized.

In one or more exemplary systems, to determine whether the patient data criterion PDC is satisfied based on the first time parameter, comprises to determine whether the second patient data PD_2, such as the second patient metric, is above a patient data threshold or within a patient data range of the patient data criterion.

In one or more exemplary systems, to determine whether the patient data criterion PDC is satisfied comprises to determine whether a difference between the first patient data PD_1 and the second patient data PD_2 based on the first time parameter. In other words, the system may be configured to determine the development or evolution of the patient status data and/or the symptoms of the patient from the moment where the first patient data was obtained to the moment where the second patient data is obtained.

In one or more exemplary systems, the first action request may comprise one or more alarms. An alarm may comprise a time limit for performing an action. In one or more exemplary systems, the first action request may comprise one or more alarms including a first alarm comprising a first time limit for performing an action. In other words, the system, such as using the electronic device and/or the server device, may be configured to determine in accordance with the first patient metric satisfying the first criterion, a first action request comprising one or more alarms. In one or more exemplary systems, the system, such as using the electronic device and/or the server device, is configured to determine in accordance with the first patient metric satisfying the first criterion and based on the hospitalization status, a first action request comprising one or more alarms. In other words, an alarm may depend on the hospitalization status of the patient.

An alarm may be based on the first patient data PD_1, the second patient data PD_2, the first patient metric, the second patient metric, and/or the hospitalization status. An alarm may be seen as an alarm (such as a request and/or a notification) to a health care professional notifying the health care professional that an action needs to be taken within a certain time period. An alarm may be determined at the electronic device and forwarded to a healthcare electronic device, such as via a global network and/or via the server device. An alarm may be determined at the server device and forwarded to a healthcare electronic device. In other words, the first action request may comprise an action request to a healthcare professional. In one or more exemplary systems, the second action request may comprise one or more alarms. The description of the one or more alarms of the first action criterion may also apply to the second action request.

For example, when a score associated with the patient, such as the first score and/or the second score, in the range of 0 to 4, the system may be configured to determine (such as generate) an alarm to a healthcare professional where the healthcare professional has to respond within 6 hours. In other words, when a score associated with the patient is indicative of a low level of acuteness, e.g., non-urgent patient status, the system may be configured to determine (such as generate) an alarm to a healthcare professional where the healthcare professional has to respond within 6 hours.

For example, when the first patient data and/or the second patient data are indicative of a blood sugar value being in the range of 10 to 25 mmol/L, such as a value being above or equal to 20 mmol/L, the system may be configured to determine (such as generate) an alarm to a healthcare professional where the healthcare professional has to respond within 6 hours.

For example, when a patient has sent a contact request not being indicative of an emergency, e.g., via the electronic device and/or the virtual epidemic centre/virtual monitoring centre, the system may be configured to determine (such as generate) an alarm to a healthcare professional where the healthcare professional has to respond within 6 hours.

For example, when a score associated with the patient, such as the first score and/or the second score, in the range of 4 to 7, the system may be configured to determine (such as generate) an alarm to a healthcare professional where the healthcare professional has to respond within 30 min. In other words, when a score associated with the patient is indicative of a medium level of acuteness, such as medium urgent patient status, the system may be configured to determine (such as generate) an alarm to a healthcare professional where the healthcare professional has to respond within 30 minutes.

For example, when the first patient data and/or the second patient data are indicative of a deterioration of symptoms, the system may be configured to determine (such as generate) an alarm to a healthcare professional where the healthcare professional has to respond within 30 minutes.

For example, when a score associated with the patient, such as the first score and/or the second score, is in the range of 7 to 10, e.g., above 7, and/or that a single parameter score is above 2, the system may be configured to determine (such as generate) an alarm to a healthcare professional where the healthcare professional has to respond within 5 minutes. In other words, when a score associated with the patient is indicative of a high level of acuteness, e.g., urgent patient status, the system may be configured to determine (such as generate) an alarm to a healthcare professional where the healthcare professional has to respond within 5 minutes.

For example, when a patient has sent a contact request being indicative of an emergency, e.g., via the electronic device and/or the virtual epidemic centre/virtual monitoring centre, the system may be configured to determine (such as generate) an alarm to a healthcare professional where the healthcare professional has to respond within 5 min.

For example, when a patient has withdrawn a consent to be home hospitalized, the system may be configured to determine (such as generate) an alarm to a healthcare professional where the healthcare professional has to respond within 5 min.

For example, when a first time parameter and/or second time parameter have been exceeded, e.g., that the time between obtaining the first patient data and the obtaining of the second patient data has exceeded a predetermined period indicated by the first time parameter, the system may be configured to determine (such as generate) an alarm to a healthcare professional where the healthcare professional has to respond within 5 min. For example, the patient may feel bad such that he/she is not able to provide patient data, the electronic device is out of order, the patient has not attended a video consultation, and/or the connection between the electronic device and the server device is not working.

In one or more exemplary systems, in accordance with a determination that the patient data criterion is satisfied, output a patient data request via the electronic device.

In other words, to output a patient data request may depend on whether the patient data has changed or not, e.g. between the first patient data and the second patient data. A patient data request may comprise a request for further patient data, such as first patient data, second patient data, and/or third patient data. A patient data request may comprise a request for patient status data and/or symptom data of a patient.

In one or more exemplary system, to output a patient data request comprises to display, e.g. on the interface of the electronic device, a user interface (e.g. a patient data request user interface, such as a further user interface), indicative of the patient data request and/or indicative of, e.g. being, a link to a portal, such as a virtual epidemic center where the patient may input further patient data.

The patient data request may comprise to display the patient data request, such as a message or information text, e.g. via the electronic device, to the patient. The message or information text may be indicative of a patient data request that the patient may react on or take action on.

In one or more example systems, the system is configured to detect a selection of the first user interface object, e.g. the user selecting or clicking in the disease monitoring/handling program or website. A user interface object may refer herein to a graphical representation of an object that is displayed on a display of the electronic device. The user interface object may be user-interactive, or selectable by a user input. For example, an image (e.g., icon), a button, and text (e.g., hyperlink) each optionally constituting a user interface object. The user interface object may form part of a widget. A widget may be seen as a mini-application that may be used by the user. The user interface object may allow the patient to input first patient data, such as patient status data and symptom data based on the patient data request. A patient data request may be output to the patient at a given time interval, e.g. based on the first time parameter.

In one or more exemplary systems, the patient data scheme comprises to obtain third patient data and/or to plan an additional video consultation with the patient. In other words, the patient data scheme may comprise, define, and/or determine a point in time, such as a date and/or a time, where the third patient data shall be obtained. In other words, the patient data scheme may comprise or define a point in time where a third screening and/or a third consultation, such as an additional video consultation, is to be performed by the patient.

For example, the patient data scheme may comprise to output a third action request including a message such as: “We recommend that you perform an additional screening and/or a consultation such as an additional video consultation, with a health care professional”.

The description of the obtaining of the first patient data and/or the obtaining of the second patient data may also apply to the obtaining of the third patient data. The description of the first patient data and/or the second patient data may apply to the third patient data. The third patient data may be obtained after the first patient data and the second patient data. In one or more exemplary systems, the patient data scheme comprises to obtain more patient data, such as fourth patient data, fifth patient data etc., depending on the need of the patient, e. g. depending on the first patient metric, the second patient metric, etc.

In one or more exemplary systems, the system is configured to transmit patient data, the second patient data, the first patient metric and/or the second patient metric to the server device.

For example, the system is configured to transmit patient data, the second patient data, the first patient metric and/or the second patient metric from the electronic device to the server device, e.g. via the electronic device (such as using the processor and the interface of the electronic device) and/or the server device (such as using the one or more processors and the interface of the server device).

In other words, the system may be configured to store the patient data, the second patient data, the first patient metric and/or the second patient metric the first conversation data, such as a speaker record, on the server device.

In one or more exemplary systems, the system is configured to store the patient data, the second patient data, the first patient metric and/or the second patient metric in the memory of the electronic device.

In one or more exemplary systems, the system is configured to store the patient data, the second patient data, the first patient metric and/or the second patient metric on the server device, such as on a memory of the server device.

In one or more exemplary systems, the system is configured to determine a patient round scheme (such as one or more of a patient round, a doctor round, and a nurse round) based on patient data. A patient round scheme may for example be seen as one or more occurrences where a healthcare employee (such as a nurse and/or a doctor) obtains information about a patient. In one or more exemplary systems, the system is configured to determine a default patient round scheme. In one or more exemplary systems, the system is configured to determine a patient round scheme based on patient data and a default patient round scheme. In one or more exemplary systems, the system is configured to determine a patient round scheme based on the first patient data and/or the second patient data, such as the first patient metric and/or the second patient metric. In one or more exemplary systems, the system is configured to determine a patient round scheme based on patient data and the hospitalization status. In one or more exemplary systems, the system is configured to determine a patient round scheme when the patient is hospitalized, e.g. home hospitalized or hospitalized in a hospital. To determine a patient round scheme may comprise to determine one or more patient rounds based on the first patient data, the second patient data, and/or the hospitalization status. To determine a patient round scheme may comprise to determine a patient round frequency, such as a number of patient rounds, a schedule of the patient rounds, a time for the patient rounds, and/or a content of the patient rounds.

For example, the patient round scheme may comprise a questionnaire comprising a plurality of questions that a health care professional may conduct.

A patient round may be a physical patient round where a health care professional visits the patient. A patient round may be a virtual patient round where a health care professional conducts the patient round virtually e.g. via the electronic device of the patient, such as at the patient, and a caretaker/health care professional electronic device at the other end. A patient round may comprise an action request, such as a first action request as described in this disclosure. A patient round may comprise indications and/or instructions from the health care professional to the patient of how to handle and/or monitor the disease to be monitored and/or handled. For example, a patient round may comprise indications and/or instructions from the health care professional to the patient of how to measure, input, and/or provide patient data, such as first patient data, second patient data, and/or third patient data. For example, a patient round may comprise indications and/or instructions from the health care professional to the patient of how to measure, input, and/or provide temperature, pulse, respiratory rate, oxygen saturation, and/or blood pressure.

A patient round scheme may be customized for the patient, for example based on the patient data, such as the patient status and/or the symptoms. For example, certain patients may have specific needs to handle the disease to be monitored and/or handled, e.g. depending on the symptoms, the age, and/or the physical condition of the patient.

In one or more exemplary systems, the patient round scheme comprises a discharge recommendation of the patient. For example, when it is determined that the first criterion is not satisfied for a first time period, the second action request comprises a request to discharge the patient. To discharge a patient may be understood as discharging a patient that no longer need to be hospitalized, such as home hospitalized or hospitalized in a hospital. In other words, a discharged patient may be understood as a patient that no longer need to receive inpatient care. A discharge recommendation of the patient may comprise a time for the discharge of the patient. For example, the discharge recommendation may comprise a number of days after which the patient may be discharged, such as after 1, 2, 3, 4, or 5 days of hospitalization.

In one or more exemplary systems, the patient round scheme comprises a discharge recommendation of the patient from an actual hospital and that the patient may instead be home hospitalized. In one or more exemplary systems, the system may be configured to update a hospitalization status based on the patient round scheme, such as based on the discharge recommendation.

In one or more exemplary systems, to obtain first patient data comprises to obtain first patient data based on first answers to a first questionnaire performed via the electronic device.

The first patient data may be based on a first patient/user input. The first patient data may comprise one or more user inputs, including a first user input.

In one or more exemplary systems, to obtain first patient data comprises to obtain first answers to a first questionnaire performed via the electronic device. The first patient metric may be based on the first answers of the patient/user to the first questions of the first questionnaire.

The first questionnaire may comprise first questions. The first questionnaire may comprise in the range from two to twenty questions, such as two, three, four, five, ten, or twenty questions.

In one or more exemplary systems, the first questionnaire comprises a plurality of first questions with associated first answers, e.g. having a first answer option configuration.

In one or more exemplary systems, the first questionnaire is based on a first question configuration. In other words, a questionnaire, such as the first questionnaire, may be generated based on a question configuration, e.g. the first question configuration. A question configuration may be indicative of a setup for a questionnaire.

In one or more exemplary systems, the system is configured to generate the first question configuration. To generate a question configuration, such as the first question configuration, may comprise selecting/generating a question from a first set of questions as the first question. To generate a question configuration, such as the first question configuration, may comprise selecting/generating a first answer option configuration, e.g. indicative of a first set of answer options associated with the first question. A question configuration may comprise a set of question identifiers and/or a set of questions. In one or more exemplary systems, the question configuration comprises a set of instruction identifiers and/or a set of instructions. An instruction may be or be indicative of a text, audio, image and/or a video that is to be presented to the patient/user. An instruction may comprise information to the patient/user.

In one or more exemplary systems, the first question configuration comprises a hierarchy comprising a first layer including a first question and a first answer option configuration indicative of a first set of answer options associated with the first question. A hierarchy comprises a first layer, a second layer and optionally a third layer, each layer comprising one or more elements. The hierarchy may comprise an order of questions and answer options. One or more rules may define the relation between a layer (or element(s) thereof) and a subjacent layer (or element(s) thereof). An element may be or comprise a question or a question identifier. An element may be or comprise an instruction or an instruction identifier. The question configuration, such as a layer of the hierarchy, may comprise one or more questions or question identifiers. The question configuration, such as a layer of the hierarchy, may comprise one or more instructions and/or instruction identifiers.

The first set of answer options may comprise at least three answer options. The first question configuration may comprise a second layer including at least two second questions associated with the first question. The first question configuration, such as the first layer of a hierarchy, may comprise a first set Qi of questions. The first set Qi of questions comprises one or more first questions or first question identifiers also denoted qi, where i is an index from 1 to n i, where n i is the number of questions in the first set of questions. Accordingly, the first set of questions may comprise a first primary question qi and/or a first secondary question. In the present context, first questions are indexed with a single index.

The first patient data may comprise answer data indicative of one or more answers being input by a patient/user, e.g. in response to one or more questions and/or instructions presented to the patient/user as part of the first questionnaire. The first patient data may comprise first answers in response to first questions. The first answers may be based on a first answer option configuration indicative of a first set of answer options associated with the first questions. In other words, the first answers may be chosen by a patient/user from the first answer option configuration. The patient/user may for example provide the first patient/user input comprising one or more answers to a questionnaire, such as a first questionnaire, e.g. performed via an electronic device. The one or more answers may be indicative of patient data, such as patient status data and/or symptom data. The first patient data may comprise text data, audio data, image data, and/or video data indicative of patient/user data.

The first patient data, such as the patient status data, may comprise static patient/user data, e.g. provided by the patient/user as first patient/user input. The first patient data may comprise patient/user identification data, such as social register number, a personal identification number, a social security number, and/or a social health security number. For example, the first patient data may comprise obtaining the first patient/user input comprising the patient/user manually inputting patient/user data such as age, gender, height, weight, health condition/health data, and/or area of residence (such as a municipality). The first patient data may comprise a first primary timestamp. The first primary timestamp may at least be indicative of a day. The first primary timestamp may be indicative of a day and time of day.

The first patient data may comprise one or more sets of first primary questions and/or first primary answers, including a first primary set of question(s) and answer(s). The first primary set of question and answer may comprise a first primary question of the first questionnaire and a first primary answer to the first primary question.

The first patient data may be indicative of patient/user data, such as comprise information about the patient such as the number of persons that lives in the same residence or home as the patient or are present at least once a day in the residence or home of the patient/user, e.g. to determine whether the patient may be home hospitalized.

The first patient data may comprise information about whether the patient/user has been in contact with a person contaminated with the disease to be monitored within the last X days (where X may be 1, 2, 3, 4, 5, 6, 7, 10, 15).

The first patient data may comprise information about whether the user/patient has been staying or resided in an area where the infection or contamination pressure is known to be high within the last X days. The first patient data may comprise information about whether the user/patient has been staying or resided in a country outside his country of residence, if yes where has the user/patient been. The first patient data may comprise patient data such as relevant anamnestic information about whether the user/patient has any relevant history of diseases or conditions that he/she knows of (e.g. chronic diseases). The first patient data may comprise information about whether the user/patient gets medication, such as treatment for hypertension, if yes whether the hypertension is well treated, e.g. having measurements below 140/90 mmHg. The first patient data may reflect the type of relevant data asked at standard hospital admission. The first patient data may comprise information about whether the user/patient has diabetes, if yes, optionally whether it is diabetes type 1 or type 2. Further, the first patient data may comprise information about whether the user/patient is being treated with insulin. The first patient data may comprise information about whether the user/patient has chronic lung disease. The first patient data may comprise information about whether the user/patient has a serious kidney disease, if yes optionally whether the user/patient is undergoing dialysis treatment. The first patient data may comprise information about whether the user/patient has been in respiratory use treatment in intensive care, e.g. within a period, such as within the last year or within the last 3 months. The first patient data may comprise information about whether the user/patient is in treatment for a cancer disease. The first patient data may comprise information about whether the user/patient is in or has been in treatment with medication that weakens his immune defense, such as adrenal cortical hormones, rheumatic medicine, psoriasis, or infection in the intestines, and if yes which medication. When the user/patient is a woman, the first patient data may comprise information about whether she is pregnant, and if yes when the term is. The first patient data may comprise information about a blood type of the user/patient.

The patient status data may be obtained via a network such as a global network, e.g. the internet. The patient status data may be obtained after the user has logged in with his/her personal login information e.g. to a social registry portal with his/her user identification data. The patient status data may comprise user data obtained from a database where user data is stored. For example, the patient status data may comprise user data obtained from a database, such as age, gender, height, weight, health condition/health data, and/or area of residence (such as a municipality) of the user. The patient status data may comprise a first timestamp. The first timestamp may at least be indicative of a day. The patient status data may comprise data obtained e.g. from other third party applications on an electronic device. For example, age, gender, height and weight data may be obtained from a health application or an operating system information database on a smartphone, where the user has typed in his/her data. In one or more exemplary systems, patient status data such as age, gender, height and weight data may be obtained via the first patient data, e.g. via one or more first questions to the patient.

The first questionnaire may comprise one or more first questions relating to the patient status data and/or the symptom data of the patient. The first questionnaire may comprise one or more first questions relating to the status of the patient status and/or the symptoms of the patient.

For example, a first primary question Fq_1 of the first questionnaire may be a question relating to a symptom of the patient: “have you had a sore throat (such as swallowing pain) in the past day(s) (e.g. past 24 hours)?”. A first primary answer option configuration may comprise three options such as yes, no, and don't know. The patient/user may then choose one of the options from the first primary answer option configuration as the first primary answer FX_i. For example, FX_i may be assigned the value or score 1 if “yes” and the value or score 0 if “No”. If the answer includes a value, such as not a yes, no, or maybe answer, the indicated value may be assigned to FX_i. If the answer includes text, such as not merely a yes, no, or maybe answer, the indicated text may be assigned to FX_i. The answer may also be selected from a list of answers, such as a list of text fields. Each text field may comprise an identifier and the identifier may be assigned to the answer FX_i.

For example, a first secondary question Fq_2 of the first questionnaire may be a question relating to a status of the patient: “have you had fever in the past day(s) (such as above 38° C.)?”. A first secondary answer option configuration may comprise three options such as yes, no, and don't know. When the answer is yes, the first secondary question Fq_2 may comprise a first secondary sub-question Fq_2_1 that may be: “if yes, can you provide a measured value of fever?”. The first secondary sub-answer option configuration may comprise a list of numerical values indicative of a measured fever temperature, such as comprised in a drop-down list, a scroll-list, or a rolling list. Alternatively, or additionally, the first secondary sub-answer option configuration may comprise a text input field, where the patient/user may type in the measured fever temperature value. The text input may then be assigned to FX_i.

For example, a first tertiary question Fq_3 of the first questionnaire may be a question relating to both a status of the patient and a symptom of the patient: “have you had shortness of breath or respiratory problems in the past day(s)? If yes, what is your respiratory rate?”. A first tertiary answer option configuration may comprise three options such as yes, no, and don't know. When the answer is yes, the first tertiary answer option configuration may comprise a list of numerical values indicative of a measured respiratory rate, such as comprised in a drop-down list, a scroll-list, or a rolling list. Alternatively, or additionally, the first tertiary answer option configuration may comprise a text input field, where the patient/user may type in the measured respiratory rate value. The text input may then be assigned to FX_i.

The first questions may change or vary depending on the disease to be handled and/or monitored, e.g. the disease in focus. In one or more exemplary methods and/or system, the disease is Covid-19.

The system comprises determining a first score based on the first patient data. The first score may be determined based on the first patient data. The first score may be determined based on one or more parameters comprised in the first patient data. In other words, the first score may be determined based on the first patient/user input. For example, the first score may be determined based on answer data indicative of answers being input by the patient/user. For example, the first score may be determined based on answer data indicative of answers being input by the patient/user during a screening (such as after having performed a screening, e.g. the first screening and/or second screening). The first score may be based on the first answers of the user to the first questions of the first questionnaire. The first score may be a weighted score. For example, the first score may be weighted based on a weighing of the obtained first patient data, e.g. on a weighing of the patient data, such as inputted patient/user data. For example, the temperature of the patient may have a larger weighting coefficient than the blood pressure or vice-versa, and the patient status data may have a larger weighting coefficient than the symptom data or vice-versa. The weighting of the first patient data may depend on the disease that is to be monitored. The first answers of the patient/user may be weighted differently depending on the provided answers and/or the first questions. The first score may be comprised in the first patient metric.

The first score may be a score out of a total number of points that may be scored. The first score may be denoted P_1. The first score may comprise the total number of points that the user has scored when answering the first questions. Each first answer from the patient/user may give a number of points assigned to the answer FX_i where i is the answer number. The first score may be expressed as a sum of values based on first answers: P_1=FX_1+FX_2+FX_3 . . . +FX_n. The number of points assigned to the answer FX_i for a question may for example be in the range from 0 to 10. The value or score 0 may be given when the answer is negative, such as a “No” answer. A positive value or score such as 1-10 may be given when the answer is positive, such as a “Yes” answer. The number of points or value given for or assigned to each answer FX_i may then depend on a weighing of the question and/or a weighting of the answer. For example, when the first primary question Fq_1 have a high importance, the first primary answer X_1 may be assigned a value or score in the range from 5 to 10 if the first primary answer X_1 is positive, e.g. a Yes answer. For example, when the first primary question Fq_1 have a medium importance, the first primary answer X_1 may be assigned the value or score in the range from 4 to 6 if the first primary answer X_1 is positive, e.g. a Yes answer. For example, when the first primary question Fq_1 have a low importance, the first primary answer X_1 may be assigned a value or score in the range from 1 to 3 if the first primary answer X_1 is positive, e.g. a Yes answer. A negative answer, such as a “No” answer may be assigned the value or score 0. In one or more embodiments, a “No” answer may be assigned a negative value or score such as in the range from −5 to −1. A “maybe” answer may be considered as a neutral answer and may be assigned a value or score in the range 0 to 1.

The first score may be given by: P_1=α_1·FX_1+α_2·FX_2+α_3·FX_3 . . . +α_n·FX_n, where α_i, i=1, . . . , n are weighting coefficients and FX_i, i=1, . . . , n are indicative of first answers. A weighting coefficient α_i may be assigned to each first answer FX_i, e.g. to each first answer option.

The first score may be given by: P_1=f1(FX_1)+f2(FX_2)+f3(FX_3) . . . +fn(FX_n), where fi, i=1, . . . , n are functions and FX_i, i=1, . . . , n are indicative of first answers. A function fi may be assigned to each answer FX_i. The functions fi may for example comprise or be based on a look-up table, a linear or non-linear function, a model, or a neural network.

In one or more exemplary systems, to determine whether the first patient metric PM_1 satisfies a first criterion C_1 may comprise to determine whether the first score is above a first threshold or within a first range of the first criterion. The first score may be seen as an early warning score for a patient. The first action request may be based on the first score. The first action request may comprise a minimum observation interval of the patient, e.g. based on the first score. The first action request may comprise a recommendation parameter for the patient, e.g. based on the first score. The recommendation parameter may comprise one or more recommendations for the patient. For example, the patient data scheme may be based on the first score. The first time parameter may be based on the first score.

For example, the first action request may comprise a minimum observation interval of the patient of every 12^{th} hour (such as in the range of every 10-15^{th} hour). For example, when the first score (such as accumulated score of the first screening/first questionnaire) is in the range of 0-1 the minimum observation interval of the patient may be every 12^{th} hour (such as in the range of every 10-15^{th} hour). For example, when the first score is in the range of 0-1 the patient data scheme may comprise a first time parameter (such as screening interval) of every 12^{th} hour between obtaining the first patient data and obtaining the second patient data. In one or more example systems, the first time parameter may be reduced for precautionary measures (such as increasing a screening interval).

For example, the first action request may comprise a minimum observation interval of the patient of every 6^{th} hour (such as in the range of every 5-7^{th} hour). For example, when the first score (such as accumulated score of the first screening/first questionnaire) is in the range of 1-2 (such as 2) the minimum observation interval of the patient may be every 6^{th} hour. For example, when the first score is in the range of 1-3, the first action request may comprise a recommendation parameter indicating to a healthcare personnel (such as a nurse) to perform an ABCD (airway, breathing, circulation, disability, and exposure) assessment of the patient.

For example, the first action request may comprise a minimum observation interval of the patient of every 4^{th} hour (such as in the range of every 3-5^{th} hour). For example, when the first score is in the range of 3-5 (such as 4) the minimum observation interval of the patient may be every 4^{th} hour. For example, when the first score is in the range of 3-5, the first action request may comprise a recommendation parameter indicating to a healthcare personnel (such as a nurse) to perform an ABCD (airway, breathing, circulation, disability, and exposure) assessment of the patient. For example, when the first score is in the range of 3-5, the first action request may comprise a recommendation parameter indicating to a healthcare personnel (such as a nurse) to contact the patient every 6^{th} hour. For example, when the first score is in the range of 3-5, the first action request may comprise a recommendation parameter indicating to a healthcare personnel (such as a nurse) to evaluate whether a doctor should be notified and/or recruited for assistance. When a doctor is recruited for assistance, the doctor may be recommended to consider a treatment plan.

For example, the first action request may comprise a minimum observation interval of the patient of every 4^{th} hour (such as in the range of every 3-5^{th} hour). For example, when the first score is in the range of 5-7 (such as 6) the minimum observation interval of the patient may be every 4^{th} hour. For example, when the first score is in the range of 5-7, the first action request may comprise a recommendation parameter indicating to a healthcare personnel (such as a nurse) to contact the patient every 6^{th} hour. For example, when the first score is in the range of 5-7, the first action request may comprise a recommendation parameter indicating to a healthcare personnel (such as a nurse) to contact and/or recruit a doctor immediately. When a doctor is recruited for assistance, the doctor may be recommended to make and document a treatment plan. For example, when the first score is in the range of 5-7, the first action request may comprise a recommendation parameter indicating to a healthcare personnel (such as a nurse) to perform an ABCD (airway, breathing, circulation, disability, and exposure) assessment of the patient.

For example, the first action request may comprise a minimum observation interval of the patient of every 1 hour. For example, when the first score is in the range of 7-8 the minimum observation interval of the patient may be every 1 hour. For example, when the first score is in the range of 5-7, the first action request may comprise a recommendation parameter indicating a request for hospitalization in a hospital (such as standard in-hospital admission). For example, when the first score is in the range of 5-7, the first action request may comprise a recommendation parameter indicating a conversion to a standard hospitalization.

For example, the first action request may comprise a minimum observation interval of the patient of every 30 minutes. For example, when the first score is above 9 the minimum observation interval of the patient may be every 30 minutes. For example, when the first score is above 9, the first action request may comprise a recommendation parameter indicating a request for hospitalization in a hospital (such as standard in-hospital admission). For example, when the first score is above 9, the first action request may comprise a recommendation parameter indicating a conversion to a standard hospitalization.

The description of the first score may also apply to the description of the second score. The description of the first action request may also apply to the description of the second action request.

In one or more exemplary systems, to obtain second patient data comprises to obtain second patient data based on second answers to a second questionnaire performed via the electronic device.

The second patient data may be based on a first patient/user input. The second patient data may comprise one or more user inputs, including a second user input.

In one or more exemplary systems, obtaining second patient data comprises obtaining second answers to a second questionnaire performed via the electronic device. The second patient metric may be based on the second answers of the patient/user to the second questions of the second questionnaire.

The second questionnaire may comprise second questions. The second questionnaire may comprise in the range from two to twenty questions, such as two, three, four, five, ten, or twenty questions.

In one or more exemplary systems, the second questionnaire comprises a plurality of second questions with associated second answers, e.g. having a second answer option configuration.

In one or more exemplary systems, the second questionnaire is based on a second question configuration. In other words, a questionnaire, such as the second questionnaire, may be generated based on a question configuration, e.g. the second question configuration. A question configuration may be indicative of a setup for a questionnaire.

In one or more exemplary systems, the system comprises generating the second question configuration. To generate a question configuration, such as the second question configuration, may comprise selecting/generating a question from a second set of questions as the second question. To generate a question configuration, such as the second question configuration, may comprise selecting/generating a second answer option configuration, e.g. indicative of a second set of answer options associated with the second question. A question configuration may comprise a set of question identifiers and/or a set of questions. In one or more exemplary systems, the question configuration comprises a set of instruction identifiers and/or a set of instructions. An instruction may be or be indicative of a text, audio, image and/or a video that is to be presented to the patient/user. An instruction may comprise information to the patient/user.

In one or more exemplary systems, the second question configuration comprises a hierarchy comprising a second layer including a second question and a second answer option configuration indicative of a second set of answer options associated with the second question. A hierarchy comprises a second layer, a second layer and optionally a third layer, each layer comprising one or more elements. The hierarchy may comprise an order of questions and answer options. One or more rules may define the relation between a layer (or element(s) thereof) and a subjacent layer (or element(s) thereof). An element may be or comprise a question or a question identifier. An element may be or comprise an instruction or an instruction identifier. The question configuration, such as a layer of the hierarchy, may comprise one or more questions or question identifiers. The question configuration, such as a layer of the hierarchy, may comprise one or more instructions and/or instruction identifiers.

The second set of answer options may comprise at least three answer options. The second question configuration may comprise a second layer including at least two second questions associated with the second question. The second question configuration, such as the second layer of a hierarchy, may comprise a second set Qi of questions. The second set Qi of questions comprises one or more second questions or second question identifiers also denoted qi, where i is an index from 1 to n i, where n i is the number of questions in the second set of questions. Accordingly, the second set of questions may comprise a second primary question qi and/or a second secondary question. In the present context, second questions are indexed with a single index.

In one or more exemplary systems, the system is configured to update the first score, based on the second patient data, e.g. based on the first time parameter, such as after the first time period. In one or more exemplary systems, the system is configured to determine a second score based on the first patient data and/or the second patient data.

The second patient data may comprise information about the patient status and/or the patient symptoms since the first patient data was obtained. The description of the first questionnaire may also apply to the second questionnaire. The description of the first questions may also apply to the second questions.

The system is configured to determine a patient metric based on the first patient metric and/or the second patient metric. The system is configured to determine a patient score based on the first patient score and/or the second patient score.

A server device for disease monitoring and/or handling is disclosed. The server device comprises a memory, an interface, and one or more processors.

The memory circuitry may be one or more of a buffer, a flash memory, a hard drive, a removable media, a volatile memory, a non-volatile memory, a random access memory (RAM), or other suitable device. In a typical arrangement, the memory circuitry may include a non-volatile memory for long term data storage and a volatile memory that functions as system memory for the processor circuitry. The memory circuitry may exchange data with the processor circuitry over a data bus. Control lines and an address bus between the memory circuitry and the processor circuitry also may be present. The memory circuitry is considered a non-transitory computer readable medium. The memory circuitry may be configured to store sensor data, and mobility data in a part of the memory.

The interface may comprise a wired interface and/or a wireless interface.

The one or more processors are optionally configured to obtain a hospitalization status of a patient.

The one or more processors are optionally configured to obtain first patient data of the patient comprising patient status data and symptom data from an electronic device. The first patient data may be obtained from an electronic device and/or a global network such as the internet, via the interface. The patient status data is indicative of one or more of temperature, pulse, respiratory rate, oxygen saturation, and blood pressure of the patient, and the symptom data is indicative of one or more symptoms of the patient.

The one or more processors are optionally configured to obtain second patient data comprising patient status data and symptom data from the electronic device. The second patient data may be obtained from an electronic device via the interface.

The one or more processors are optionally configured to determine a first patient metric based on the first patient data.

The one or more processors are optionally configured to determine whether the first patient metric satisfies a first criterion.

The one or more processors are optionally configured in accordance with the first patient metric satisfying the first criterion, to transmit a first action request to the electronic device.

The first action request data may be indicative of a first action request. The processor of the electronic device may be configured to generate a first action request based on the first action request data received from the server device. The processor of the electronic device may be configured to output the generated first action request to the patient/user via the interface.

An electronic device for disease monitoring and/or handling is disclosed. The electronic device comprises a memory, an interface (such as one or more interfaces), and a processor (such as one or more processors). The memory circuitry may be one or more of a buffer, a flash memory, a hard drive, a removable media, a volatile memory, a non-volatile memory, a random access memory (RAM), or other suitable device. In a typical arrangement, the memory circuitry may include a non-volatile memory for long term data storage and a volatile memory that functions as system memory for the processor circuitry. The memory circuitry may exchange data with the processor circuitry over a data bus. Control lines and an address bus between the memory circuitry and the processor circuitry also may be present. The memory circuitry is considered a non-transitory computer readable medium. The memory circuitry may be configured to store sensor data, and mobility data in a part of the memory.

The interface may comprise a wired interface and/or a wireless interface. The interface may comprise one or more displays including a first display.

The electronic device may be configured to act as a user device that a user may use for disease monitoring. The electronic device may comprise a computer device (such as a mobile phone, a smartphone, a cell phone, a tablet, and/or a computer such as a laptop or a stationary computer).

The processor is configured to obtain a hospitalization status of a patient from a server device, e.g. a server device according to this disclosure.

The processor is optionally configured to obtain first patient data of the patient comprising patient status data and symptom data via the interface. The first patient data may optionally be obtained from the server device and/or a global network such as the internet, via the interface. The patient status data is indicative of one or more of temperature, pulse, respiratory rate, oxygen saturation, and blood pressure of the patient, and the symptom data is indicative of one or more symptoms of the patient.

The processor is optionally configured to obtain second patient data comprising patient status data and symptom data via the interface. The second patient data may be obtained from a server device via the interface.

The electronic device may be configured to obtain/receive one or more user inputs such as a first patient/user input and/or a second patient/user input. The first patient/user input and/or the second patient/user input may be received via the interface, such as a user interface.

The first display of the electronic device may be configured to detect the first patient/user input and/or the second patient/user input such as a touch input from the user, for example when the first display comprises a touch-sensitive display. The first patient/user input and/or the second patient/user input may comprise a contact on the touch sensitive display. A touch-sensitive display may provide a first user interface and/or a second user interface (such as an input interface) and an output interface between the electronic device and the patient/user. The processor of the electronic device may be configured to receive and/or send electrical signals from/to touch-sensitive display. A touch-sensitive display may be configured to display visual output to the user. The visual output optionally includes graphics, text, icons, video, and any combination thereof (collectively termed “graphics”). For example, some, most, or all of the visual output may be seen as corresponding to user-interface objects.

The processor of the electronic device may be configured to display, on the first display, one or more user interfaces, such as user interface screens, including a first user interface and/or a second user interface. A user interface may comprise one or more, such as a plurality of user interface objects. For example, the first user interface may comprise a first primary user interface object and/or a first secondary user interface object. A second user interface may comprise a second primary user interface object and/or a second secondary user interface object. A user interface may be referred to as a user interface screen.

An input, such as the first patient/user input and/or the second patient/user input, may comprise a touch (e.g. a tap, a force touch, a long press), and/or movement of contact (e.g. a swipe gesture, e.g. for toggling). The movement on contact may be detected by a touch sensitive surface, e.g. on the first display of the electronic device. Thus, the first display may be a touch sensitive display. The first input (such as first patient/user input), such as first input and/or second input, may comprise a lift off. A patient/user input, such as the first patient/user input and/or the second patient/user input, may comprise a touch and a movement followed by a lift off.

The processor is optionally configured to transmit, e.g. via the interface, the first patient data to the server device.

The processor is optionally configured to obtain, e.g. via the interface, a first action request from the server device.

The processor is optionally configured to output the first action request, such as to the patient, via the interface.

Also disclosed is a method for disease monitoring and/or handling. The method comprises obtaining a hospitalization status of a patient. The method comprises obtaining first patient data of the patient comprising patient status data and symptom data. The patient status data is indicative of one or more of temperature, pulse, respiratory rate, oxygen saturation, and blood pressure of the patient. The symptom data is indicative of one or more symptoms of the patient. The method comprises determining a first patient metric based on the first patient data. The method comprises determining whether the first patient metric satisfies a first criterion. The method comprises in accordance with the first patient metric satisfying the first criterion, outputting a first action request.

It is to be understood that a description of a feature in relation to a system, an electronic device, and/or a server device, is also applicable to the corresponding feature in a method(s).

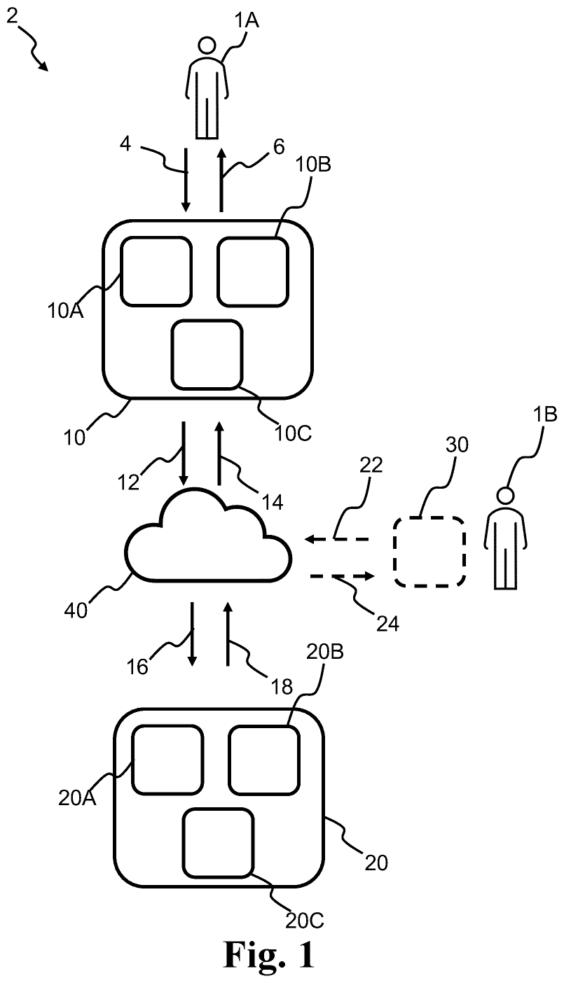

FIG. 1 schematically illustrates an exemplary system for disease monitoring and/or handling according to the present disclosure. The system 2 for disease monitoring comprises an electronic device 10 and a server device 20. The electronic device 10 comprises a memory 10A, one or more interfaces 10B, and a processor 10C. The server device 20 comprises a memory 20A, one or more interfaces 20B, and one or more processors 20C. A user/patient 1A may provide first patient data, second patient data, and/or third patient data to the electronic device 10, e.g. by performing a questionnaire, such as a first questionnaire, a second questionnaire, and/or a third questionnaire via the electronic device 10. The user/patient 1A may perform the questionnaire via a first display of the one or more interfaces 10B of the electronic device 10. The electronic device 10 may be configured to act as a user device that the user/patient 1A may use for disease monitoring/handling. The processor 10C is configured to obtain 14 a hospitalization status of a patient from the server device 20, e.g. via a network 40 such as a global network, e.g. the internet.

The processor 10C is configured to obtain 4 first patient data comprising patient status data and symptom data via the interface 10B. Optionally, the processor 10C is configured to obtain 4 second patient data comprising patient status data and symptom data via the interface 10B. The processor 10C is configured to transmit 12 the first patient data and/or the second patient data to the server device 20, e.g. via a network 40 such as a global network, e.g. the internet. The processor 10C is configured to obtain 14 a first action request data from the server device 20, e.g. via the network 40. The processor 10 is configured to output 6 the first action request to the user/patient 1A via the interface 10B.

Optionally, the processor 10C is configured to obtain 4 first patient data via performance of a first questionnaire via the interface 10B. Optionally, the processor 10C is configured to obtain 4 second patient data via performance of a second questionnaire via the interface 10B. Optionally, the electronic device 10 is configured to obtain 14 first patient data and/or second patient data, via the network 40, e.g. from the server device 20.

The one or more processors 20C are configured to obtain 16 a hospitalization status of a patient.

The one or more processors 20C are configured to obtain 16 first patient data comprising patient status data and symptom data from the electronic device 10. The first patient data may be obtained from the electronic device 10 and/or the network 40, via the interface 20B. Optionally, the one or more processors 20C are configured to obtain 16 second patient data comprising patient status data and symptom data from the electronic device 10, e.g. via the network 40. The patient status data is indicative of one or more of temperature, pulse, respiratory rate, oxygen saturation, and blood pressure of the patient, and the symptom data is indicative of one or more symptoms of the patient. The one or more processors 20C are configured to determine a first score based on the first patient data. The one or more processors 20C are configured to determine a first patient metric based on the first patient data. The one or more processors 20C are configured to determine whether the first patient metric satisfies a first criterion. The one or more processors 20C are configured in accordance with the first patient metric satisfying the first criterion, to transmit 18 a first action request to the electronic device 10, e.g. via the network 40.

The processor 10C of the electronic device 10 may be configured to generate a first action request based on the first action request data received from the server device 20. The processor 10C of the electronic device 10 may be configured to output 6 the generated first action request to the user/patient 1A via the interface 10B.

Optionally, the system 2 comprises a health care professional/caretaker electronic device 30 configured to operate as a user device for a caretaker 1B, such as a doctor, a nurse, or the like. The caretaker electronic device 30 may be configured to transmit 22 first action request data to the electronic device 10 and/or the server device 20, via the network 40. For example, the caretaker 1B may transmit 22 a first action request based on the first action request data to the user/patient 1A via the caretaker electronic device 30, the network 40, and the electronic device 10. The caretaker electronic device 30 may be configured to receive/obtain 24 the first patient data, the second patient data, and/or the third patient data from the electronic device 10 and/or the server device 20, e.g. via a network 40 such as a global network, e.g. the internet. The caretaker electronic device 30 may be configured to be used in relation to a virtual patient round application and/or a virtual epidemic centre as described in this disclosure. The caretaker electronic device 30 may allow a health care professional to conduct one or more screenings of the patient and/or a consultation of the patient. The caretaker electronic device 30 may allow a health care professional to conduct one or more questionnaires, such as a first questionnaire and/or a second questionnaire, with the patient 1A via the network 40.

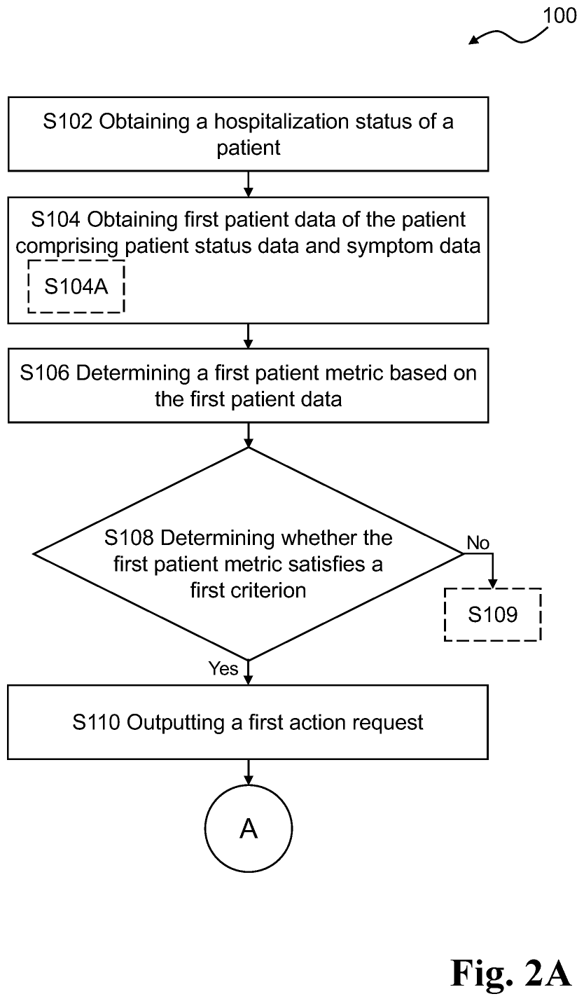

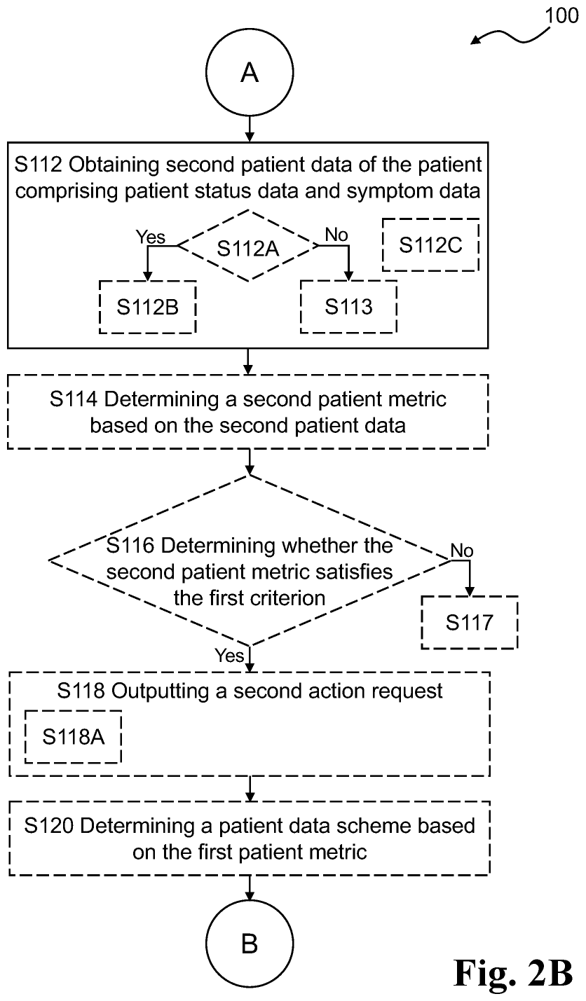

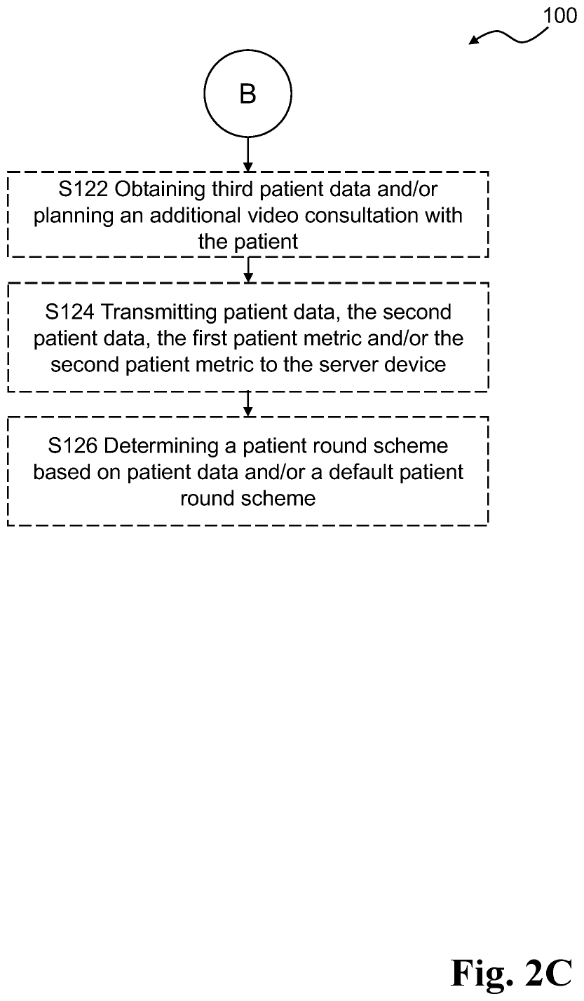

The processor 10C and/or the processor 20C are optionally configured to perform any of the operations disclosed in FIGS. 2A-2C (such as any one or more of S104A, S109, S112A, S112B, S112C, S113, S114, S116, S117, S118, S118A, S120, S122, S124, S126). The operations of the electronic device and/or the server device may be embodied in the form of executable logic routines (for example, lines of code, software programs, etc.) that are stored on a non-transitory computer readable medium (for example, memory circuitry 10A, 20A) and are executed by processor circuitry 10C, 20C).

Furthermore, the operations of the electronic device and/or the server device as disclosed herein (such as electronic device 10 and/or server device 20) may be considered a method that the electronic device and/or the server device is configured to carry out. Also, while the described functions and operations may be implemented in software, such functionality may also be carried out via dedicated hardware or firmware, or some combination of hardware, firmware and/or software.

FIGS. 2A-B is a flow diagram of an exemplary method for disease monitoring and/or handling according to the present disclosure.

The method 100 comprises obtaining S102 a hospitalization status of a patient.

The method 100 comprises obtaining S104 first patient data of the patient comprising patient status data and symptom data. The patient status data is indicative of one or more of temperature, pulse, respiratory rate, oxygen saturation, and blood pressure of the patient, and the symptom data is indicative of one or more symptoms of the patient.

In one or more exemplary methods, obtaining S104 first patient data comprises obtaining S104A first patient data based on first answers to a first questionnaire performed via the electronic device.

The method 100 comprises determining S106 a first patient metric based on the first patient data.

The method 100 comprises determining S108 whether the first patient metric satisfies a first criterion. The method 100 comprises in accordance with the first patient metric satisfying the first criterion, outputting S110 a first action request.

When it is not determined that the first patient metric satisfies the first criterion, refraining S109 from outputting a first action request, such as outputting instead “We currently do not recommend a hospitalization”.

In one or more exemplary methods, the method 100 comprises obtaining S112 second patient data of the patient comprising patient status data and symptom data. The patient status data is indicative of one or more of temperature, pulse, respiratory rate, oxygen saturation, and blood pressure of the patient, and the symptom data is indicative of one or more symptoms of the patient.

In one or more exemplary methods, the method 100 comprises determining S114 a second patient metric based on the second patient data.

In one or more exemplary methods, the method 100 comprises determining S116 whether the second patient metric satisfies the first criterion.

In one or more exemplary methods, the method 100 comprises in accordance with the second patient metric satisfying the first criterion, outputting S118 a second action request.

In one or more exemplary methods, when it is not determined that the second patient metric satisfies the first criterion, the method 100 comprises refraining S117 from outputting a first action request, such as outputting instead “We currently do not recommend a hospitalization”.

In one or more exemplary methods, the method 100 comprises determining S120 a patient data scheme based on the first patient metric. In one or more exemplary methods, obtaining S112 the second patient data is based on the patient data scheme.

In one or more exemplary methods, obtaining S112 second patient data comprises determining S112A whether a patient data criterion based on the first time parameter is satisfied.

In one or more exemplary methods, in accordance with a determination S112A that the patient data criterion is satisfied, the method 100 comprises outputting S112B a patient data request via the electronic device.

In one or more exemplary methods, when it is not determined that the patient data criterion is satisfied, the method 100 comprises refraining S113 from outputting a patient data request, such as outputting instead “No further patient data is needed”.

In one or more exemplary methods, the method 100 comprises obtaining S122 third patient data and/or planning an additional video consultation with the patient.

In one or more exemplary methods, the method 100 comprises transmitting S124 patient data, the second patient data, the first patient metric and/or the second patient metric to the server device.

In one or more exemplary methods, the method 100 comprises determining S126 a patient round scheme based on patient data and/or a default patient round scheme.

In one or more exemplary methods, outputting S118 a second action request comprises determining S118A the second action request based on the determination of whether the second patient metric satisfies the first criterion.

In one or more exemplary methods, obtaining S112 second patient data comprises obtaining S112C second patient data based on second answers to a second questionnaire performed via the electronic device.

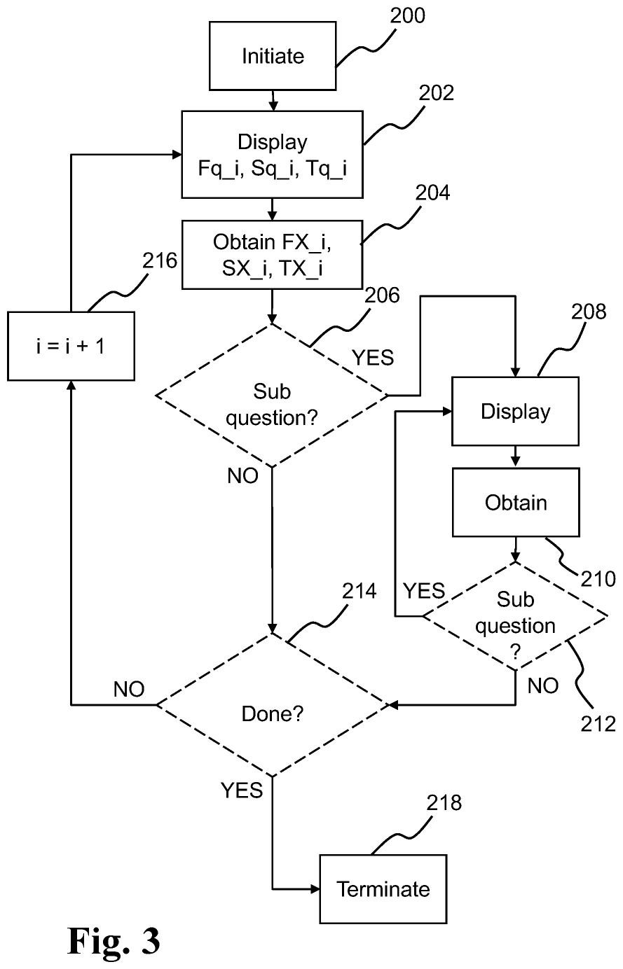

FIG. 3 is a flow diagram illustrating an example on how to obtain first patient data (such as patient status data and/or symptom data), see e.g. S104, S104A of FIG. 2A, second patient data, see e.g. S112, S112C of FIG. 2B, and/or third patient data, see e.g. S122 of FIG. 2C. To generate a question configuration, such as the first question configuration, may comprise selecting/generating a question from a first set of questions as the first question. To generate a question configuration, such as the first question configuration, the second primary question configuration, and/or the second secondary configuration may comprise selecting/generating a first question. The obtain first patient data, second patient data and/or third patient data may comprise to initiate 200 a questionnaire, such as a first questionnaire, a second questionnaire, and/or a third questionnaire. The first questionnaire, the second questionnaire, and/or the third questionnaire may be initiated by providing the first question, i.e. i=1. The first question may be a first primary question Fq_1 of the first questionnaire Fq_i, a second primary question Sq_1 of the second questionnaire Sq_i, and/or a third primary question Tq_1 of the third questionnaire Tq_i. To generate a question configuration, may comprise selecting/generating a first answer option configuration, e.g. indicative of a first set of answer options associated with the first question. To obtain first patient data, second patient data and/or third patient data may comprise to display 202 a first question Fq_i, Sq_i, and/or Tq_i to the user, e.g. via an electronic device, e.g. by displaying a text field with the first question on the user interface. To obtain first patient data, second patient data and/or third patient data, may comprise to obtain 204 a first answer FX_i, SX_i, and/or TX_i to the first question. To obtain first patient data, second patient data and/or third patient data may then comprise to determine/check 206 whether a sub-question exists based on the first question Fq_i, Sq_i, and/or Tq_i and/or on the first answer FX_i, SX_i, and/or TX_i. When a sub-question exists, a sub-question Fq_i_m, Sq_i_m, Tq_i_m may be displayed 208 to the user, e.g. via the electronic device. To obtain first patient data, second patient data and/or third patient data may then comprise to obtain 210 a sub-answer FX_i_m, SX_i_m, and/or TX_i_m to the sub-question Fq_i_m, Sq_i_m, Tq_i_m. To obtain first patient data, second patient data and/or third patient data may then comprise to determine/check 212 whether further sub-questions exists based on the first question Fq_i, Sq_i, Tq_i, the first answer FX_i, SX_i, and/or TX_i, and/or the first sub-answer FX_i_m, SX_i_m, and/or TX_i_m to the sub-question. To obtain first patient data, second patient data and/or third patient data may then comprise to determine/check 214 whether the questionnaire is done, e.g. whether there are further questions in the questionnaire or not. When it is determined that the questionnaire is done, to obtain first patient data, second patient data and/or third patient data may comprise to terminate 218 the questionnaire, e.g. to store the first patient data, second patient data and/or third patient data. When it is not determined that the questionnaire is done, to obtain first patient data, second patient data and/or third patient data comprises to provide the next question of the questionnaire that has been initiated to the user by increment 216 the question number by, i.e. i=i+1 and displaying the next question. The flow diagram is then repeated until the questionnaire is done.

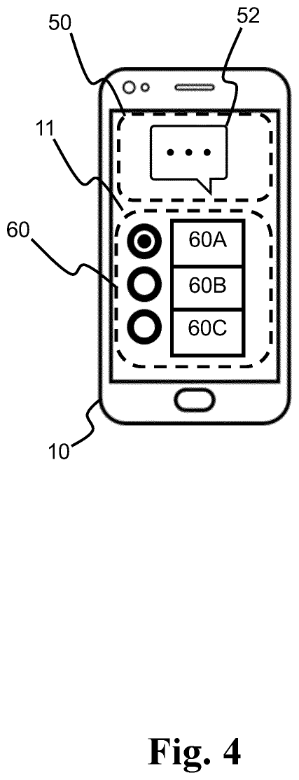

FIG. 4 schematically illustrates a user interface of an electronic device according to the present disclosure, such as the electronic device 10. The electronic device 10 comprises a first display 11 configured to display one or more user interfaces and to receive patient/user input via the one or more user interfaces, such as first patient data, second patient data, and/or third patient data. The one or more user interfaces comprise a first question user interface 50 and a first answer user interface 60 based on a first questionnaire. The processor of the electronic device 10 may be configured to display, on the first display 11 the first question user interface 50 and the first answer user interface 60. The first question user interface 50 comprises a user interface object 52 comprising a first question text. The first answer user interface 60 comprises three user interface objects 60A, 60B, 60C. The three user interface objects comprising a first user interface object 60A, a second user interface object 60B, and a third user interface object 60C. The three user interface objects may be indicative of three different answer options, e.g. the first user interface object 60A being indicative of answer option “yes”, the second user interface object 60B being indicative of answer option “no”, and the third user interface object 60C being indicative of answer option “don't know”. As may be seen on FIG. 4, the user has provided a first primary user input indicative of a selection of the first user interface object 60A, i.e. there is a dot in the first circle. In other words, the user has selected the answer “yes” to the first question. In one or more exemplary systems, to obtain the first patient data, such as the outcome of a first screening and/or a first consultation (e.g. performed on the electronic device 10), may comprise to obtain the first patient input comprising the patient manually inputting patient data such as patient status data and/or symptom data, on the electronic device (e.g. in the first answer user interface 60).

The use of the terms “first”, “second”, “third” and “fourth”, “primary”, “secondary”, “tertiary” etc. does not imply any particular order but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Memory may be one or more of a buffer, a flash memory, a hard drive, a removable media, a volatile memory, a non-volatile memory, a random access memory (RAM), or other suitable device. In a typical arrangement, memory may include a non-volatile memory for long term data storage and a volatile memory that functions as system memory for the processor. Memory may exchange data with processor over a data bus. Memory may be considered a non-transitory computer readable medium.

Memory may be configured to store information (such as information indicative of the first patient data, the second patient data, the first patient metric, the second patient metric, the first action request, the second action request, the patient data scheme, the first time parameter, and/or the patient round scheme) in a part of the memory.

Furthermore, the labelling of a first element does not imply the presence of a second element and vice versa.

It may be appreciated that FIGS. 1-4 comprise some modules or operations which are illustrated with a solid line and some modules or operations which are illustrated with a dashed line. The modules or operations which are comprised in a solid line are modules or operations which are comprised in the broadest example embodiment. The modules or operations which are comprised in a dashed line are example embodiments which may be comprised in, or a part of, or are further modules or operations which may be taken in addition to the modules or operations of the solid line example embodiments. It should be appreciated that these operations need not be performed in order presented. Furthermore, it should be appreciated that not all of the operations need to be performed. The exemplary operations may be performed in any order and in any combination.

Other operations that are not described herein can be incorporated in the example operations. For example, one or more additional operations can be performed before, after, simultaneously, or between any of the described operations.

Certain features discussed above as separate implementations can also be implemented in combination as a single implementation. Conversely, features described as a single implementation can also be implemented in multiple implementations separately or in any suitable sub-combination. Moreover, although features may be described above as acting in certain combinations, one or more features from a claimed combination can, in some cases, be excised from the combination, and the combination may be claimed as any sub-combination or variation of any sub-combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value.

The various exemplary methods, devices, and systems described herein are described in the general context of method steps processes, which may be implemented in one aspect by a computer program product (such as an application on an electronic device), embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly, to be regarded in an illustrative rather than restrictive sense. The claimed invention is intended to cover all alternatives, modifications, and equivalents.

### LIST OF REFERENCES

1A user
1B caretaker
2 system
4 obtain from user
6 output to user
10 electronic device
11 first display
10A memory
10B interface
10C processor
12 transmit to server device
14 obtain from server device
16 obtain from electronic device
18 transmit to electronic device
20 server device
20A memory
20B interface
20C one or more processors
22 transmit to electronic device and/or server device
24 obtain from electronic device and/or server device
30 caretaker electronic device
40 network
50 first question user interface
52 user interface object
60 first answer user interface
60A first user interface object
60B second user interface object
60C third user interface object
100 method for disease monitoring
200 initiate
202 display
204 obtain
206 determine
208 display
210 obtain
212 determine
214 determine
216 increment
218 terminate
S102 obtaining a hospitalization status of a patient
S104 obtaining first patient data of the patient comprising patient status data and symptom data
S104A obtaining first patient data based on first answers to a first questionnaire performed via the electronic device
S106 determining a first patient metric based on the first patient data
S108 determining whether the first patient metric satisfies a first criterion
S109 refraining from outputting a first action request
S110 outputting a first action request
S112 obtaining second patient data of the patient comprising patient status data and symptom data
S112A determining whether a patient data criterion based on the first time parameter is satisfied
S112B outputting a patient data request via the electronic device
S112C obtaining second patient data based on second answers to a second questionnaire performed via the electronic device
S113 refraining from outputting a patient data request
S114 determining a second patient metric based on the second patient data
S116 determining whether the second patient metric satisfies the first criterion
S117 refraining from outputting a first action request
S118 outputting a second action request
S118A determining the second action request based on the determination of whether the second patient metric satisfies the first criterion
S120 determining a patient data scheme based on the first patient metric
S122 obtaining third patient data and/or planning an additional video consultation with the patient
S124 transmitting patient data, the second patient data, the first patient metric and/or the second patient metric to the server device
S126 determining a patient round scheme based on patient data and/or a default patient round scheme

1. A system for disease monitoring and/or handling, the system comprising an electronic device and a server device, wherein the system is configured to:
obtain, via the electronic device and/or the server device, a hospitalization status of a patient;
obtain, via the electronic device and/or the server device, first patient data of the patient comprising patient status data and symptom data, the patient status data being indicative of one or more of temperature, pulse, respiratory rate, oxygen saturation, blood pressure, consciousness, and blood sugar of the patient, and the symptom data being indicative of one or more symptoms of the patient;
determine, using the electronic device and/or the server device, a first patient metric based on the first patient data;
determine, using the electronic device and/or the server device, whether the first patient metric satisfies a first criterion; and
in accordance with the first patient metric satisfying the first criterion, output a first action request.

2. The system according to claim 1, wherein the system is configured to:
obtain second patient data of the patient comprising patient status data and symptom data, the patient status data being indicative of one or more of temperature, pulse, respiratory rate, oxygen saturation, and blood pressure of the patient and the symptom data being indicative of one or more symptoms of the patient;
determine a second patient metric based on the second patient data;
determine whether the second patient metric satisfies the first criterion; and
in accordance with the second patient metric satisfying the first criterion, output a second action request.

3. The system according to claim 2, wherein the system is configured to determine a patient data scheme based on the first patient metric, and wherein to obtain the second patient data is based on the patient data scheme.

4. The system according to claim 3, wherein the patient data scheme comprises a first time parameter indicative of a time between the obtaining of the first patient data and the obtaining of the second patient data.

5. The system according to claim 4, wherein to obtain second patient data comprises to:
determine whether a patient data criterion, based on the first time parameter, is satisfied; and
in accordance with a determination that the patient data criterion is satisfied, output a patient data request via the electronic device.

6. The system according to claim 3, wherein the patient data scheme comprises to obtain third patient data and/or to plan an additional video consultation with the patient.

7. The system according to claim 2, wherein the system is configured to transmit patient data, the second patient data, the first patient metric and/or the second patient metric to the server device.

8. The system according to claim 1, wherein the hospitalization status of the patient comprises information on whether the patient is hospitalized or not, and when the patient is hospitalized, information on whether the patient is hospitalized in a hospital or home hospitalized.

9. The system according to claim 2, wherein the system is configured to determine a patient round scheme based on patient data and/or a default patient round scheme.

10. The system according to claim 1, wherein to output a first action request comprises to determine the first action request based on the determination of whether the first patient metric satisfies the first criterion.

11. The system according to claim 1, wherein to output a first action request comprises to determine the first action request based on the determination of whether the first patient metric satisfies the first criterion and based on the hospitalization status.

12. The system according to claim 2, wherein to output a second action request comprises to determine the second action request based on the determination of whether the second patient metric satisfies the first criterion.

13. The system according to claim 9, wherein the patient round scheme comprises a discharge recommendation of the patient.

14. The system according to claim 2, wherein the first action request and/or the second action request comprises one or more of a request to contact a doctor, a request to activate a virtual monitoring center, a request for home hospitalization, and a request for hospitalization in a hospital.

15. The system according to claim 1, wherein to obtain first patient data comprises to obtain first patient data based on first answers to a first questionnaire performed via the electronic device.

16. The system according to claim 2, wherein to obtain second patient data comprises to obtain second patient data based on second answers to a second questionnaire performed via the electronic device.

17. The system according to claim 1, wherein to determine the first patient metric is based on the first patient data and the hospitalization status.

18. The system according to claim 1, wherein the first action request comprises one or more alarms including a first alarm comprising a first time limit for performing an action.

19. Server device for disease monitoring and/or handling, the server device comprising a memory; an interface; and one or more processors, wherein the one or more processors are configured to:
obtain, via the interface, a hospitalization status of a patient;
obtain, via the interface, first patient data of the patient comprising patient status data and symptom data from an electronic device, the patient status data being indicative of one or more of temperature, pulse, respiratory rate, oxygen saturation, blood pressure, consciousness, and blood sugar of the patient, and the symptom data being indicative of one or more symptoms of the patient;
determine a first patient metric based on the first patient data;
determine whether the first patient metric satisfies a first criterion; and
in accordance with the first patient metric satisfying the first criterion, transmit, via the interface, a first action request to the electronic device.

20. Electronic device for disease monitoring and/or handling, the electronic device comprising a memory; an interface; and a processor, wherein the processor is configured to:
obtain, via the interface, a hospitalization status of a patient from a server device;
obtain, via the interface, first patient data of the patient comprising patient status data and symptom data via the interface, the patient status data being indicative of one or more of temperature, pulse, respiratory rate, oxygen saturation, blood pressure, consciousness, and blood sugar of the patient, and the symptom data being indicative of one or more symptoms of the patient;
transmit, via the interface, the first patient data to the server device;
obtain a first action request from the server device; and
output the first action request via the interface.
